(19) United States
(12) Patent Application Publication
(10) Pub. No.: US 2010/0196275 A1
(43) Pub. Date: Aug. 5, 2010

(54) Methods and products for neutralizing the harmful effects of combustion products

(76) Inventor: Moon-Shong Tang, Upper saddle River, NJ (US)

Correspondence Address: KLAUBER & JACKSON, 411 HACKENSACK AVENUE, HACKENSACK, NJ, 07601, US

(21) Appl. No.: 12/311,232

(22) PCT Filed: Sep. 21, 2007
(86) PCT No.: PCT/US2007/020446

§ 371 Date: Dec. 7, 2009

Related U.S. Application Data
(60) Provisional application No. 60/846,540, filed on Sep. 22, 2006.
(60) Provisional application No. 60/848,233, filed on Sep. 29, 2006.

(51) Int. Cl. A61K 49/00; C12N 5/02; C12Q 1/68; C40B 30/04; B01D 15/04; G01N 27/26; G01N 33/559; B01D 53/34; B01D 53/44; B01D 53/46; B01D 53/72; A24D 3/06; A62B 23/02

(52) U.S. Cl. 424/9.1; 435/375; 435/6; 506/9; 210/660; 204/450; 204/461; 422/177; 210/690; 423/210; 423/245.1; 131/332; 128/205.27

(57) ABSTRACT

The invention relates to the role of Acr-DNA adducts in p53 mutagenesis in CS-related lung cancer. The distribution of Acr-DNA adducts was mapped at the sequence level in the p53 gene of lung cells using the UvrABC incision method in combination with ligation-mediated PCR. It was determined that Acr preferentially binds at methylated CpG sites. Also, Acr can greatly reduce the DNA repair capacity for damage induced by benzo(a)pyrene diol epoxide. Together these results suggest that Acr is a major etiological agent for CS-related lung cancer and that it contributes to lung carcinogenesis through DNA damage and inhibition of DNA repair. Methods and compositions are provided for either removing an exogenous toxic agent from a combustion product or for preventing the toxic or mutagenic effect of these toxic agents on cells and tissues. Methods are also provided for screening for candidate compounds that protect cells from toxic combustion products.

### GOVERNMENT RIGHTS CLAUSE

The research leading to the present invention was supported by National Cancer Institute Grant number CA114541. Accordingly, the Government has certain rights in the invention.

### FIELD OF THE INVENTION

This invention relates generally to methods for removing, neutralizing, or sequestering an exogenous toxic agent from a liquid or a gaseous material, wherein the exogenous toxic agent is reactive with a p53 gene or a fragment thereof. Methods are also provided for preventing or inhibiting the mutagenic effect of such toxins on cells or tissues. The invention also relates to methods of screening for a candidate compound that prevents the binding of a p53 tumor suppressor inhibitor to a p53 molecule, wherein the binding results in abrogation of the tumor suppressing activity or function of the p53 molecule. The invention also relates to methods of removing a toxic aldehyde, in particular, acrolein, from combustion products, in particular, smoke generated by tobacco products or smoke generated by heating cooking oil to a high temperature.

### BACKGROUND

The use of tobacco products, especially smoking, is associated with an increased incidence of lung cancer, as well as other types of cancer. It is also known that individuals who smoke have a higher incidence of emphysema and cardiovascular disease. It is also becoming more apparent that the combustion of cooking oils leads to the generation of toxic combustion products, which may also lead to an increase in cancerous conditions in individuals exposed to these toxic combustion products. (J. Natl. Cancer Institute (1995); 87:836-841).

Tobacco smoke is a complex mixture which includes numerous chemical compounds and particulates which to a major extent are responsible for the higher rate of cancers associated with individuals who smoke. However, these same chemical compounds contribute to the enjoyment of smoking. Among the many compounds present in tobacco smoke are the purported addictive component nicotine, compounds responsible for flavor, and those either proven harmful or believed to be harmful to human health. Tobacco smoke contains chemical toxins such as carbon monoxide and hydrogen cyanide, and known carcinogens such as formaldehyde and hydrazine. Specific compounds in tobacco smoke may fall into more than one of these categories, such as those responsible for flavor. The search for a method of reducing the exposure of smokers to these toxic compounds without affecting the flavor of smoke while maintaining nicotine delivery has been sought for many decades.

The harmful effects of such combustion products, including tobacco use, and principally cigarette smoking, derive from the delivery to the body of toxic compounds present in tobacco or from cooking oils and volatilized during combustion. These include gaseous compounds, such as carbon monoxide, hydrogen cyanide, ammonia, and formaldehyde, and others that are volatilized in tobacco smoke, such as benzene, hydrazine, and aniline. Collectively, the solid material which may be condensed from tobacco smoke is known as tar. Several compounds in smoke and tar are classified as carcinogens: benzene, 2-naphthylamine, 4-aminobiphenyl, and the radioactive element polonium-210. Others are considered probably human carcinogens, such as formaldehyde, hydrazine, N-nitrosodimethylamine, N-nitrosodiethylamine, N-nitrosopyrrolidine, benzo[a]pyrene, N-nitrosodiethanolamine, and cadmium.

Numerous methods and devices to reduce or remove toxic components from tobacco and more importantly from the combustion products of tobacco have been proposed and constructed. For example, a porous filter may be provided as a first line trap for harmful components, and this is interposed between the smoke stream and the mouth. This type of filter, which is often composed of cellulose acetate, acts both mechanically and by adsorption to trap a certain fraction of the tar present in smoke. While this type of filter is present on most cigarettes available, it still allows a significant amount of harmful compounds to pass into the mouth, as evidenced by the fact that the cancer incidence remains high in spite of the use of filtered cigarettes.

Many have attempted improvements in the effectiveness afforded by these mechanical-type filters such as those described above by including a means for chemically trapping disagreeable and harmful components present in smoke.

For example, U.S. Pat. No. 5,076,294 provides a filter element containing an organic acid, such as citric acid, which reduces the harshness of the smoke. A significant body of art focuses on removing formaldehyde, a prevalent component of tobacco smoke with an established and adverse toxicological profile.

U.S. Pat. No. 4,300,577 describes a filter comprising an absorptive material plus an amine-containing component which removes aldehydes and hydrogen cyanide from tobacco smoke.

U.S. Pat. No. 5,009,239 describes a filter element treated with polyethyleneimine modified with an organic acid, to remove aldehydes from tobacco smoke.

U.S. Pat. No. 4,246,910 describes a filter impregnated with alkali ferrate compounds, or activated carbon or alumina impregnated with potassium permanganate, for removing hydrogen cyanide from tobacco smoke. Control of the delivery of tar, nicotine, formaldehyde and total particulate matter was afforded by a filter element containing zinc thiocyanate, sarcosine hydrochloride, zinc chloride, ferrous bromide, lithium bromide, or manganese sulfate, as describe in U.S. Pat. No. 4,811,745.

Inclusion of L-ascorbic acid in a filter material to remove aldehydes is disclosed in U.S. Pat. No. 4,753,250, while U.S. Pat. No. 5,060,672 also describes a filter for specifically removing aldehydes, such as formaldehyde, from tobacco smoke by providing a combination of an enediol compound, such as dihydroxyfumaric acid or L-ascorbic acid, together with a radical scavenger of aldehydes, such as oxidized glutathione or urea, or a compound of high nucleophilic activity, such as lysine, cysteine, 5,5-dimethyl-1,3-cyclohexanedione, or thioglycolic acid. Such filters, however, have not been shown to reduce the harmful effects of tobacco smoke, and have yet to demonstrate adequate consumer acceptance or commercial viability. Furthermore, many of the agents used in the above-mentioned filters, such as organic acids, will trap nicotine and interfere with its delivery to the smoker.

Accordingly, there is still a need for determining improved methods of reducing the toxic effects of combustions products, such as from smoking, while retaining the enjoyment benefits of smoking tobacco. Furthermore, there is also a need for identifying the agent in tobacco smoke and other combustion products that may play a key role in carcinogenesis or mutagenesis induced by such toxic combustion products. More importantly, there is still a need for improved methods for removal or neutralization of these agents from air or from tobacco smoke, as well as improved methods for protecting cells and tissues from the toxic effects of these combustion products. These needs are addressed by the present application.

All publications, patent applications, patents and other reference material mentioned are incorporated by reference in their entirety. In addition, the materials, methods and examples are only illustrative and are not intended to be limiting. The citation of references herein shall not be construed as an admission that such is prior art to the present invention.

### SUMMARY OF THE INVENTION

In its broadest aspect, the present invention provides methods for preventing or inhibiting mutagenesis or carcinogenesis in a cell or tissue, wherein the cell or tissue contains a p53 gene, and wherein the mutagenesis or carcinogenesis is caused by an exogenous toxic agent. The methods provide for contacting the cell or tissue in vitro or in vivo with a composition comprising a second agent that blocks the reactivity of the exogenous toxic agent with a p53 gene, or a target sequence within the p53 gene, or a fragment thereof within the p53 gene, wherein such reactivity results in an increase in DNA adduct formation, and wherein the contacting prevents the binding of the exogenous toxic agent to the p53 gene, or the target sequence in the p53 gene, or a fragment thereof, thereby reducing or inhibiting DNA adduct formation. In one particular embodiment, the target sequence or fragment thereof is present in exons 5, 7 and 8 of the p53 gene. In another embodiment, the exogenous toxic agent is removed from a liquid or from a gaseous material using a filter that reacts with the toxic agent, thereby preventing it from reaching its target molecule within the cell or tissue. In another particular embodiment, the toxic agent is acrolein, and it is removed by filtering the liquid or gaseous material through a filter comprised of a proteinaceous agent or a nucleic acid or fragment thereof for which the acrolein shows specificity. Other non-proteinaceous or non-nucleic acid materials may be used, for example, small organic molecules containing chemical groups that react with acrolein, may be attached to the filter, or impregnated within the filter, such that upon exposure of the filter to a liquid or gas containing acrolein, the acrolein will react with the material within the filter or attached to the filter, and will not be available for entry into the cell or tissue. In another particular embodiment, the filter may be attached to a tobacco smoking device, such as a cigarette, or it may be a filter used to adsorb toxic materials from room air, such as combustion products generated during cooking foods at high temperatures, or to remove the toxic combustion products from second-hand smoke.

Another aspect of the invention provides a method for removing, neutralizing, or sequestering an exogenous toxic agent from a liquid, wherein said exogenous toxic agent is reactive with a p53 gene or a fragment thereof, comprising contacting said liquid with a composition comprising the nucleic acid encoding the p53 tumor suppressor, or a target sequence, or a fragment thereof within the p53 gene, that is reactive with, or binds to, the exogenous toxic agent.

Another aspect of the invention provides a method for removing, neutralizing, or sequestering an exogenous toxic agent from a gaseous material, wherein said exogenous toxic agent is reactive with a p53 gene or a fragment thereof, the method comprising contacting the gaseous material containing the exogenous toxic agent with a filter comprising a polymeric material derivatized with the nucleic acid encoding the p53 gene, or the p53 gene product, or a target sequence, or a fragment thereof within the p53 gene, that is reactive with, or binds to, the exogenous toxic agent.

In one particular embodiment, the polymeric material is selected from the group consisting of cellulose, starch and agarose.

In another particular embodiment, the exogenous toxic agent is an aldehyde. In a more particular embodiment, the aldehyde is acrolein.

In another particular embodiment, the target sequence within the p53 gene is an unmethylated or methylated nucleobase, or an unmethylated or methylated dinucleotide, or a combination thereof. In yet another particular embodiment, the methylated nucleobase is a cytosine. In yet another particular embodiment, the methylated dinucleotide is CpG. In yet another particular embodiment, the methylated CpG is present in either the promoter region or the coding region of the p53 gene. In a more particular embodiment, the methylated CpG is found in codons 152, 154, 156, 157, 158 of exon 5; in codon 248 of exon 7, and in codons 273 and 282 of exon 8 of the p53 gene. In another more particular embodiment, the unmethylated nucleobase is found in codon 249 in exon 7 of the p53 gene.

Another aspect of the invention provides a method for screening for a candidate compound that prevents the binding of a p53 tumor suppressor inhibitor to a p53 molecule, wherein the binding results in abrogation of the tumor suppressing activity or function of the p53 molecule. The method comprises the following steps:

(a) contacting the p53 molecule, or fragments thereof, or genomic DNA comprising the p53 molecule, with a candidate compound in the presence or absence of a known inhibitor, wherein the p53 molecule has the nucleic acid sequence of SEQ ID NO: 1 or SEQ ID NO: 3 and/or the amino acid sequence of SEQ ID NO: 2 or SEQ ID NO: 4; and

(b) determining the level of p53 expression or activity/function in the presence or absence of the candidate compound;

wherein the candidate compound is considered to be effective if the level of p53 expression or activity/function is higher in the presence of the candidate compound as compared to in the absence of the candidate compound.

Any in vitro method for measuring the level of expression of the p53 gene or gene product may be used, as known to those skilled in the art. For example, measurements may be done by one or more of the following methods: reverse transcription-polymerase chain reaction (RT-PCR), real time PCR, northern blot analysis, in situ hybridization, cDNA microarray, electrophoretic gel analysis, an enzyme immunoassay (ELISA assays), a Western blot, a dotblot analysis, a protein microarray, a flow cytometric technique and proteomics analysis.

The candidate compound that is found to be effective by virtue of any of these methods may be further tested in vivo. In one embodiment, the method further comprises:

(c) treating a tumor bearing animal with a candidate compound capable of increasing the level of expression or function of the p53 molecule, and assessing the effect of the candidate compound on the growth, progression or metastasis of a tumor,

wherein the tumor arises as a result of loss of function of the p53 molecule or wherein the p53 molecule is mutated as a result of exposure of a cell in the animal to a mutagen; wherein a candidate compound effective at inhibiting the growth, progression, or metastasis of a tumor in said animal is identified as a positive candidate compound.

In one particular embodiment, the fragment of the p53 molecule is derived from exons 5, 7, or 8 of the p53 gene. In another particular embodiment, the fragments contain one or more unmethylated or methylated cytosines, and the methylated cytosines are present in a CpG dinucleotide. Furthermore, the methylated cytosines are present in codons 152, 154, 156, 157, and 158 of exon 5, in codon 248 of exon 7 and in codons 273 and 282 of exon 8, whereas the unmethylated cytosine is present in codon 249 of exon 7.

In another particular aspect, the invention provides a method of screening for a candidate compound capable of inhibiting the binding of a mutagenic agent to a p53 molecule, or to genomic DNA containing a p53 molecule, or to a fragment, nucleobase or dinucleotide derived therefrom, the method comprising the following steps:
(a) contacting the p53 molecule, or a fragment, nucleobase or dinucleotide derived therefrom, or genomic DNA containing the p53 gene, with a known mutagenic agent in the absence and presence of a candidate compound, wherein the p53 molecule is:
   (i) a DNA corresponding to SEQ ID NO: 1 or SEQ ID NO: 3, and wherein the nucleic acid fragment, nucleobase, or dinucleotide is obtained from exons 5, 7 or 8 of the p53 gene of SEQ ID NO: 1 or SEQ ID NO: 3;
   (ii) a protein comprising SEQ ID NO: 2 or SEQ ID NO: 4, or a fragment derived therefrom;
   (iii) a nucleic acid comprising a sequence hybridizable to SEQ ID NO: 1 or SEQ ID NO: 3 or a complement thereof under conditions of high stringency, or a protein comprising a sequence encoded by said hybridizable sequence; or
   (iv) a nucleic acid at least 90% homologous to SEQ ID NO: 1 or SEQ ID NO: 3 or a complement thereof as determined using an NBLAST algorithm or a protein encoded thereby;
(b) determining whether or not the candidate compound blocks the mutagenic effect of the known mutagenic agent on the p53 molecule;

wherein the candidate compound is considered a positive candidate compound If it blocks the mutagenic effect of the known mutagenic agent on DNA adduct formation.

As noted above, one particular embodiment provides for assessing the effect of a known mutagenic agent on the p53 molecule by assessing the formation of one or more DNA adducts in the p53 gene or a fragment thereof. In another more particular embodiment, the formation of a DNA adduct is measured by a UVR-BC/LMPCR method.

The method may further comprise a step of assessing or confirming the activity of the candidate in an in vivo tumor model. The method comprises:

(c) treating a tumor bearing animal with the candidate compound and assessing the effect of the candidate compound on the growth, progression or metastasis of the tumor, wherein the tumor arises as a result of loss of function of the p53 molecule or wherein the p53 molecule is mutated as a result of exposure of a cell in the animal to a mutagen;

wherein a candidate compound effective at inhibiting the growth, progression, or metastasis of a tumor in the animal is identified as a positive candidate compound.

In one particular embodiment, the p53 fragment contains one or more unmethylated or methylated cytosines. In another particular embodiment, the methylated cytosines are present in a CpG dinucleotide. In yet another more particular embodiment, the methylated cytosines are present in codons 152, 154, 156, 157, and 158 of exon 5, in codon 248 of exon 7 and in codons 273 and 282 of exon 8, and the unmethylated cytosine is present in codon 249 of exon 7.

Another aspect of the invention provides a method for reducing the level of a toxic aldehyde, for example, acrolein, present in air containing combustion products by passing the air through a filter element capable of removing the toxic aldehyde, eg. acrolein, present in the air, wherein the filter element comprises a polymer derivatized with a proteinaceous agent, or a nucleic acid molecule, or fragment thereof for which the toxic aldehyde is specific, or to which it binds. In one embodiment, the sequence is obtained from the nucleic acid encoding the p53 tumor suppressor, or a fragment thereof, that is reactive with, or binds to, acrolein. In another particular embodiment, the nucleic acid sequence obtained from the p53 tumor suppressor gene, or a fragment thereof that reacts with, or binds to, acrolein, is an unmethylated or methylated nucleobase, or an unmethylated or methylated dinucleotide, or a combination thereof. In yet another more particular embodiment, the methylated nucleobase is a cytosine. In yet another more particular embodiment, the methylated dinucleotide is CpG. In yet another more particular embodiment, the methylated CpG is present in either the promoter region or the coding region of the p53 gene. In yet another more particular embodiment, the methylated CpG is found in codons 152, 154, 156, 157, and 158 in exon 5; in codon 248 in exon 7 and in codons 273 and 282 in exon 8, of the p53 gene. In yet another more particular embodiment, the unmethylated nucleobase is found in codon 249 in exon 7 of the p53 gene.

In yet another more particular embodiment, the air comprises the vapor generated by volatilization of cooking oils and greases.

In yet another more particular embodiment, the air comprises smoke generated by mainstream tobacco smoke or from second-hand tobacco smoke.

In yet another more particular embodiment, the mainstream tobacco smoke retains nicotine content and desirable flavor components after passage through said filter.

In yet another more particular embodiment, the polymer is selected from the group consisting of cellulose, starch, agarose, and combinations thereof.

In yet another more particular embodiment, the method is used to filter air in a tobacco smoke-generating device or in a tobacco smoke-containing environment selected from the group consisting of a cigarette, free-standing cigarette filter, pipe, cigar, air ventilation filter, gas mask, and face mask.

Another aspect of the invention provides for a filter for removing acrolein from a combustion product, wherein the filter comprises a polymeric substrate to which is attached a chemical group reactive with acrolein.

In one particular embodiment, the polymeric substrate is selected from the group consisting of cellulose, starch and agarose.

In another particular embodiment, the chemical group reactive with acrolein is found on an agent selected from the group consisting of an amino acid, a peptide or protein, a nucleic acid, an oligonucleotide, a polynucleotide, a dinucleotide, a methylated dinucleotide, a nucleobase, a methylated nucleobase, methylated CpG, and any other synthetic or naturally occurring compound reactive with an aldehyde group, and combinations thereof.

In yet another particular embodiment, the nucleic acid comprises the nucleotide sequence set forth in SEQ ID NO: 1.

In yet another particular embodiment, the methylated dinucleotide is CpG. In yet another particular embodiment, the methylated nucleobase is a cytosine. In yet another particular embodiment, the nucleic acid is obtained from exons 5, 7, or 8 of the p53 gene. In yet another particular embodiment, the CpG is found at a location selected from the group consisting of codons 152, 154, 156, 157, and 158 in exon 5; codon 248 in exon 7, codons 273 and 282 in exon 8 of the p53 gene, and combinations thereof.

In yet another aspect, the invention provides for a device for reducing the level of acrolein present in air containing combustion products, wherein the device comprises a filter element through which air passes, wherein the filter element is capable of removing acrolein present in the air, and wherein the filter element comprises a polymer derivatized with an agent containing an aldehyde reactive group.

In one particular embodiment, the device filters smoke generated from frying or grilling food products, or from mainstream tobacco smoke, or from second-hand tobacco smoke.

In another particular embodiment, mainstream tobacco smoke retains nicotine content and desirable flavor components after passage through the filter.

In yet another particular embodiment, the polymer is selected from the group consisting of cellulose, starch, agarose, and combinations thereof.

Another aspect of the invention provides for a device for reducing the level of acrolein present in air containing mainstream or secondary tobacco combustion products, or acrolein generated by heating cooking oils, wherein the device comprises a filter element through which air passes, and wherein the filter element capable of removing acrolein present in the air comprises an agent selected from the group consisting of an amino acid, a peptide or protein, a nucleic acid, an oligonucleotide, a polynucleotide, a dinucleotide, a methylated dinucleotide, a nucleobase, a methylated nucleobase, methylated CpG, and any other synthetic or naturally occurring compound reactive with an aldehyde group, and combinations thereof.

In one particular embodiment, the nucleic acid comprises the nucleotide sequence set forth in SEQ ID NO: 1, or a fragment thereof.

In another particular embodiment, the methylated dinucleotide is CpG.

In yet another particular embodiment, the methylated nucleobase is a cytosine.

In another particular embodiment, the nucleic acid is obtained from exons 5, 7, or 8 of the p53 gene.

In yet another particular embodiment, the methylated CpG is found at a location selected from the group consisting of codons 152, 154, 156, 157, and 158 in exon 5; codon 248 in exon 7, codons 273 and 282 in exon 8 of the p53 gene, and combinations thereof.

In yet another particular embodiment, the device is selected from the group consisting of a cigarette, a free-standing cigarette filter, a pipe, a cigar, an air ventilation filter, an air conditioner filter, a gas mask, and a face mask.

Other objects and advantages will become apparent to those skilled in the art from a review of the following description which proceeds with reference to the following illustrative drawings.

### BRIEF DESCRIPTION OF THE DRAWINGS

FIG. 1.(A) Chemical structure of acrolein and Acr-dG adducts and (B) Identification of ³²P-labeled Acr-DNA adducts by 2D TLC. Acr-modified DNA isolated from Acr-treated human cells and Acr-treated purified genomic DNA were digested with phosphodiesterase and nuclease P1, labeled with □-³²P-ATP and subjected to 2 D TLC, as described in the Materials and Methods. The standard Acr-dG adducts were obtained by reaction of Acr (0.2 M) with dGMP

(2 mM) and then labeled with ³²P-ATP, and the major Acr-dG 3 adducts are indicated by circles. a) DNA isolated from control cells, b) Acr-modified genomic DNA, c) DNA from Acr-treated cells, d) Acr-modified dGMP, and e) mixtures of c) and d).

FIG. 2. Acr-dG and BPDE-dG adduct distribution in exons 5, 7 and 8 of the p53 gene of normal human lung cells treated with Acr (A) and BPDE (B). In (A) NHBE cells and NHLF were treated with 20 μM Acr for 6 h, and in (B) NHBE cells were treated with 1 μM BPDE for 30 min. Genomic DNA was then isolated, the DNA adduct distribution was mapped by the UvrABC/LMPCR method and the DNA was separated by electrophoresis. A/G and T/C are Maxam and Gilbert reaction products (26). (C) Comparisons of the frequency of Acr-dG adduct distribution along the p53 gene in NHBE cells with the frequency of the p53 mutations in CS-related lung cancer (IARC, p53 Mutation Database 2006).

FIG. 3. The effect of ⁵C cytosine methylation at CpG sites on Acr-dG adduct formation. Cytosines at CpG sites of (A) 5′-³²P-labeled exon 5 and (B) 3′-³²P-labeled exon 7 of p53 DNA fragments were methylated by SssI CpG methylase, and the DNA fragments with and without methylation treatment were modified with Acr (30 μM, 10 h incubation), treated with UvrABC nucleases and separated by electrophoresis as previously described (25). A/G and T/C are Maxam and Gilbert reaction products, T/*C represents Maxam and Gilbert reaction products from methylated DNA fragments. *C represents the methylated cytosine, and the codon number of the bands corresponding to CpG sites is indicated with *.

FIG. 4. Inhibition of the repair of BPDE-DNA adducts in human cells by Acr. (A) Repair inhibition determined by host cell reactivation assay. BPDE-modified luciferase reporter and unmodified β-galactosidase plasmids were co-transfected into NHLF treated with different concentrations of Acr for 1 h, and luciferase and □-galactosidase activities were measured 20 h post transfection. The relative repair capacity was calculated as the percentage of the relative luciferase activity of the plasmids in Acr-treated cells as compared to untreated cells after normalization of the transfection frequency with β-galactosidase activity. (B-D) Repair inhibition determined by in vitro DNA repair synthesis assay. BPDE-modified pUC18 and unmodified pBR322 plasmids were used as DNA substrates for in vitro DNA repair synthesis assay. In (B) NHLF were treated with different concentrations of Acr for 1 h and the cell extracts were used for repair assay. In (C) different concentrations of Acr were added directly into cell extracts prepared from untreated NHLF immediately before the start of repair assay. The upper panel is a photograph of an ethidium bromide-stained gel, and the lower panel is an autoradiograph of the same gel. In (D) the relative repair capacity was calculated as the percentage of the repair activity in Acr-treated samples to untreated samples. The data represent three independent experiments, and the error bar represents the standard deviation.

FIG. 5. Mutational spectrum of the human p53 gene in human lung cancers of cigarette smokers and nonsmokers (IARC, P53 mutation database 2006). Red bars represent mutations occurring at codons with CpG sequences and black bars represent mutations occurring at codons without CpG sequences.

FIG. 6. UvrABC nuclease cuts Acr-dG adducts specifically and quantitatively. A) Supercoiled pGEM plasmid modified with different concentrations of Acr was cut with UvrABC. The resultant DNAs were separated in a 1% agarose gel. OC: open circular plasmid, CCC: covalently closed circle plasmid. B) The ³²P-3′-end labeled p53 exon 7 DNA fragment and C) ³²P-5′-end labeled p53 exon 5 DNA fragment were modified with Acr and then reacted with UvrABC. The resultant DNAs were separated by gel electrophoresis.

FIG. 7. Kinetics of UvrABC cutting on unmethylated and methylated ³²P-3′-end labeled p53 DNA fragments. The ³²P-3′-end labeled p53 exon 7 DNA fragments (A) with and (B) without 5C cytosine methylation at CpG-sequences were modified with Acr and cut with UvrABC for different times. Codons (245 and 248) with methylated cytosines were indicated by asterisks. In (A) and (B) a typical autoradiograph is presented, and in (C) the kinetics of UvrABC incision at different sequences (9 well-separated bands are quantified, with bars represent the range of relative cutting) are presented. The rate constant of UvrABC incision for Acr-dG formed at different sequences including -CpG- sites in which 5C cytosine are methylated are similar, if not identical, indicating that the extent of UvrABC incision at different sequences represents the level of Acr-dG formation.

FIG. 8. Acr-dG adduct distribution in exons 5, 7 and 8 of the p53 gene in genomic DNA modified with Acr. Genomic DNA isolated from untreated NHBE was modified with 30 μM acrolein for 10 h, the DNA adduct distribution was mapped by the UvrABC/LMPCR method, and the resultant DNA were separated by electrophoresis. A/G and T/C (lanes 1 & 2) are Maxam and Gilbert reaction products. Lanes 3 to 5 are genomic DNA isolated from NHBE cells.

FIG. 9. Depiction of human p53 gene and relevant exons denoting the target regions for binding of acrolein to p53.

FIG. 10. Human p53 gene and relevant binding sites for acrolein to p53 in regions rich in CpG.

FIG. 11. Mouse p53 gene and relevant binding sites for acroelin to p53 in regions rich in CpG.

All publications, patent applications, patents and other reference material mentioned are incorporated by reference in their entirety. In addition, the materials, methods and examples are only illustrative and are not intended to be limiting. The citation of references herein shall not be construed as an admission that such is prior art to the present invention.

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M.J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### DEFINITIONS

The terms used herein have the meanings recognized and known to those of skill in the art, however, for convenience and completeness, particular terms and their meanings are set forth below.

As referred to herein, the “p53 gene” is one of the most studied and well-known tumor suppressor genes. The p53 gene plays a key role in cellular stress response mechanisms by converting a variety of different stimuli, for example, DNA damage, deregulation of transcription or replication, and oncogene transformation, into cell growth arrest or apoptosis (T. M. Gottlieb et al., Biochem. Biophys. Acta, 1287, p. 77 (1996)). When p53 is activated, it causes cell growth arrest or a programmed, suicidal cell death, which in turn acts as an important control mechanism for genomic stability. In particular, p53 controls genomic stability by eliminating genetically damaged cells from the cell population, and one of its major functions is to prevent tumor formation. p53 is inactivated in a majority of human cancers (A. J. Levine et al., Br. J. Cancer, 69, p. 409 (1994) and A. M. Thompson et al., Br. J. Surg., 85, p. 1460 (1998)). When p53 is inactivated, abnormal tumor cells are not eliminated from the cell population, and are able to proliferate. A “loss of function of p53” refers to any means by which the p53 gene is inactivated, whereby such inactivation of p53 is associated with a high rate of tumor progression and a resistance to cancer therapy.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. Thus, the term encompasses DNA, RNA, as well as, their constituent nucleotides, nucleosides, bases (also referred to as nucleobases) and derivatives thereof. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5N to 3N direction along the nontranscribed strand of DNA (i.e., the strand having a sequence homologous to the mRNA). A derivative may include for example, nucleic acid modified by exposure to a mutagen or carcinogen. A nucleic acid may be a dinucleotide, oligonucleotide, or polynucleotide. It may be a methylated nucleobase or dinucleotide or polynucleotide containing one or more methylated bases. It may include natural or synthetic forms, including that prepared by genetic engineering techniques. For example, a “recombinant DNA molecule” is a DNA molecule that has undergone a molecular biological manipulation.

By “homologous” is meant a same sense nucleic acid which possesses a level of similarity with the target nucleic acid within reason and within standards known and accepted in the art. With regard to PCR, the term “homologous” may be used to refer to an amplicon that exhibits a high level of nucleic acid similarity to another nucleic acid, e.g., the template cDNA. As is understood in the art, enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplified nucleic acid (i.e., amplicon) need not be completely identical in every nucleotide to the template nucleic acid.

Two DNA sequences are “substantially homologous” or “substantially similar” when at least about 50% (preferably at least about 75%, and most preferably at least about 90, or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al.; DNA Cloning, Vols. I & II; Nucleic Acid Hybridization.

Similarly, two amino acid sequences are “substantially homologous” or “substantially similar” when greater than 50% of the amino acids are identical, or functionally identical. Preferably, the similar or homologous sequences are identified by alignment using, for example, the GCG (Genetics Computer Group, Program Manual for the GCG Package, Version 7, Madison, Wis.) pileup program.

Thus, “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the sequences of the present invention.

Thus, the term “percent identical” or “percent sequence identity” refers to sequence identity between two amino acid sequences or between two nucleotide sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

The terms “protein”, “polypeptide” and “peptide” are used interchangeably herein when referring to a gene product and indicates a molecular chain of amino acids linked through covalent and/or noncovalent bonds. The terms do not refer to a specific length of the product. Thus, peptides, oligopeptides and proteins are included within the definition of polypeptide. The terms include post-expression modifications of the polypeptide, for example, glycosylations, acetylations, phosphorylations and the like. In addition, protein fragments, analogs, mutated or variant proteins, fusion proteins and the like are included within the meaning of polypeptide.

The terms “synthetic peptide” or “synthetic polypeptide” or “synthetic protein” are used interchangeably and refer to polymeric forms of amino acids of any length, which may be chemically synthesized by methods well-known to the skilled artisan. These synthetic peptides are useful in various applications.

The term “polynucleotide” as used herein means a polymeric form of nucleotides of any length, either ribonucleotides or deoxyribonucleotides. This term refers only to the primary structure of the molecule. Thus, the term includes double- and single-stranded DNA, as well as, double- and single-stranded RNA. It also includes modifications, such as methylation or capping, and unmodified forms of the polynucleotide. The terms “polynucleotide,” “oligomer,” “oligonucleotide,” and “oligo” are used interchangeably herein.

“Complementary” is understood in its recognized meaning as identifying a nucleotide in one sequence that hybridizes (anneals) to a nucleotide in another sequence according to the rule A→T, U and C→G (and vice versa) and thus “matches” its partner for purposes of this definition. Enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplicon need not be completely matched in every nucleotide to the target or template RNA.

Procedures using conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art. (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc.)

“Fragment” refers to either a protein or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide.

A “conservative amino acid substitution” refers to the substitution of one or more of the amino acid residues of a protein with other amino acid residues having similar physical and/or chemical properties. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point. Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

The terms “prevent”, “preventing”, and “prevention,” or “inhibit”, or “inhibiting”, as used herein, are intended to refer to a decrease in the occurrence of a mutation, or a disease resulting from such mutation, for example, such as that which may occur by way of exposure or contact of a cell, tissue or organism, with a mutagenizing agent. A cancer or hyperproliferative disease is an example of a disease that would result from exposure of a cell, tissue or organism to a mutagenizing agent. Such agents are any that are known to those skilled in the art to result in DNA adduct formation or inhibition of a DNA repair mechanism, which would aid in the elimination of, for example, thymidine dimers, or other forms of nucleic acid adducts, such as those described herein. The prevention may be complete, e.g., the total absence of mutagenesis, or the absence of a disease resulting from the exposure to a mutagen. The prevention may also be partial, such that the amount of disease is less than that which would have occurred without the present invention. For example, the extent of disease using the methods of the present invention may be at least 10%, preferably at least 20%, at least 30%, at least 40%, at least 50%, at least 60%, at least 70%, at least 80%, at least 90%, or at least 99% less than the amount of disease that would have occurred without the present invention.

A “DNA adduct” is a covalent modification of DNA bases by chemicals that can alter the structure and, in turn, the biological processing of the DNA by cellular proteins governing replication, transcription and repair. If not repaired or repaired incorrectly, these modifications may eventually lead to mutations and ultimately cancer, especially if the adduct is located in an oncogene or tumor suppressor gene, such as the p53 gene described herein.

The terms “removing”, “neutralizing” or “sequestering” are used interchangeably in the present application, and refer to either the partial, or complete elimination of an exogenous toxic agent from a liquid or gas, or the blocking of the toxic effect of an exogenous toxic agent on a cell, tissue or organism, by competing for binding sites on the toxic agent. Such agents that may be useful for removing, neutralizing, or sequestering an exogenous toxic agent may be an organic compound that has reactive sites that react chemically with the toxic agent, or a proteinaceous material, or a nucleic acid molecule, which also may react with particular chemical groups on an exogenous toxic agent, thus preventing subsequent binding of the exogenous toxic agent to a cell, tissue or organism.

The term “exogenous toxic agent” refers to any type of molecule, or compound, which is not naturally found in the body, but which may be found external to the body in any form, eg. solid, liquid or gaseous, which, upon exposure to a cell, tissue, or organism, results in a toxic or mutagenic effect.

The term “target sequence” as used herein, refers to any particular site, or sites on a nucleobase, or a nucleic acid molecule, or a specific gene, or a polypeptide or protein encoded by such gene, to which an exogenous toxic agent may bind. For example, the work presented herein refers to particular acrolein-DNA adducts that are formed. Acrolein, which is exemplary of an “exogenous toxic agent” appears to bind preferentially to CpG sites in the p53 gene. More specifically, the preferential sites appear to be methylated cytosines. Furthermore, specific codons, as described herein, appear to be the target for acrolein. Thus, these sites are considered to be the “target sequence” for acrolein.

A “polymeric material” as used herein refers to any material in polymeric form, which can be used in the preparation of filters, such filters to be used for the removing, neutralizing or sequestering of an exogenous toxic agent. The polymeric materials may be selected from the group consisting of cellulose, starch or agarose, or any combination thereof.

“CpG” refers to phosphodiester-linked cytosine and guanine. “CpG islands” are regions of DNA near and in approximately 40% of promoters of mammalian genes. They are regions where a large concentration of phosphodiester-linked cytosine and guanine pairs exist. The “p” in CpG represents that they are phosphodiester-linked. Unlike CpG sites in the coding region of a gene, in most instances, the CpG sites in the CpG islands are unmethylated if genes are expressed. The usual formal definition of a CpG island is a region with at least 200 by and with a GC percentage that is greater than 50% and with an observed/expected CpG ratio that is greater than 0.6.

“Candidate compound” or “test compound” refers to any compound or molecule that is to be tested, and more particularly for the present invention, for its ability to inhibit the binding of a mutagenic agent to p53. As used herein, the terms, which are used interchangeably, refer to biological or chemical compounds such as simple or complex organic or inorganic molecules, peptides, proteins, peptidomimetics, peptide mimics, antibodies, nucleic acids (DNA or RNA), including oligonucleotides, polynucleotides, dinucleotides, nucleobases, antisense molecules, small interfering nucleic acid molecules, such as siRNA or shRNA molecules, carbohydrates, lipoproteins, lipids, small molecules and other drugs. A vast array of compounds can be synthesized and tested using the methods described herein. In addition, various natural sources can provide compounds for screening, such as plant or animal extracts, and the like. Compounds can be tested singly or in combination with one another. Agents or candidate compounds can be randomly selected or rationally selected or designed. As used herein, an agent or candidate compound is said to be “randomly selected” when the agent is chosen randomly without considering the specific interaction between the agent and the target compound and target site. As used herein, an agent is said to be “rationally selected or designed”, when the agent is chosen on a nonrandom basis which takes into account the specific interaction between the agent and the target site and/or the conformation in connection with the agent's action.

“Mainstream tobacco smoke” refers to the smoke that is inhaled from a smoking device, such as a cigarette or pipe.

“Second-hand smoke” refers to smoke generated by the smoking device and inhaled by nonsmokers who are in the vicinity of a smoker.

The “UVR-BC/LMPCR” refers to a method using the UvrABC nuclease incision in combination with the ligation-mediated polymerase chain reaction technique as described in the following references: Hu, W. et al. Biochemistry (2003), 42: 10012-10023; Feng, Z. et al. J. Natl. Cancer Institute, (2002), 94(20): 1527-1536; Feng, Z. et al, Carcinogenesis, (2003), 24(4):771-778.

“Diagnosis” or “screening” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

“Screening for a candidate compound” refers to any one or more methods useful for identifying a compound that has the desired activity.

The “animal” of the invention may be a human or non-human mammal. The “non-human mammal” of the invention include mammals such as rodents, including rats, mice and guinea pigs and non-human primates, and sheep, goats, rabbits, dogs, cats, cows, chickens, amphibians, reptiles, etc.

### General Description

The tumor suppressor gene p53 is frequently mutated in human cancers (Olivier, M., Eeles, R., Hollstein, M., Khan, M.-A., Harris, C.-C., Hainaut, P. (2002) Hum. Mutat. 19, 607-614; Greenblatt, M.-S., Bennett, W.-P., Hollstein, M., Harris, C.-C. (1994) Cancer Res. 54, 4855-4878), and its mutational patterns often bear the fingerprints of the etiological carcinogens. Most notably it has been found that more than 50% of alfatoxin B1-associated liver cancers have mutations in codon 249 of the p53 gene and p53 mutations are concentrated at contiguous pyrimidines in sunlight-associated skin cancers (Hsu, I.-C., Metcalf, R.-A., Sun, T., Welsh, J.-A., Wang, N.-J., Harris, C.-C. (1991) Nature 350, 427-428; Brash, D.-E. (1997) Trends Genet. 13, 410-414).

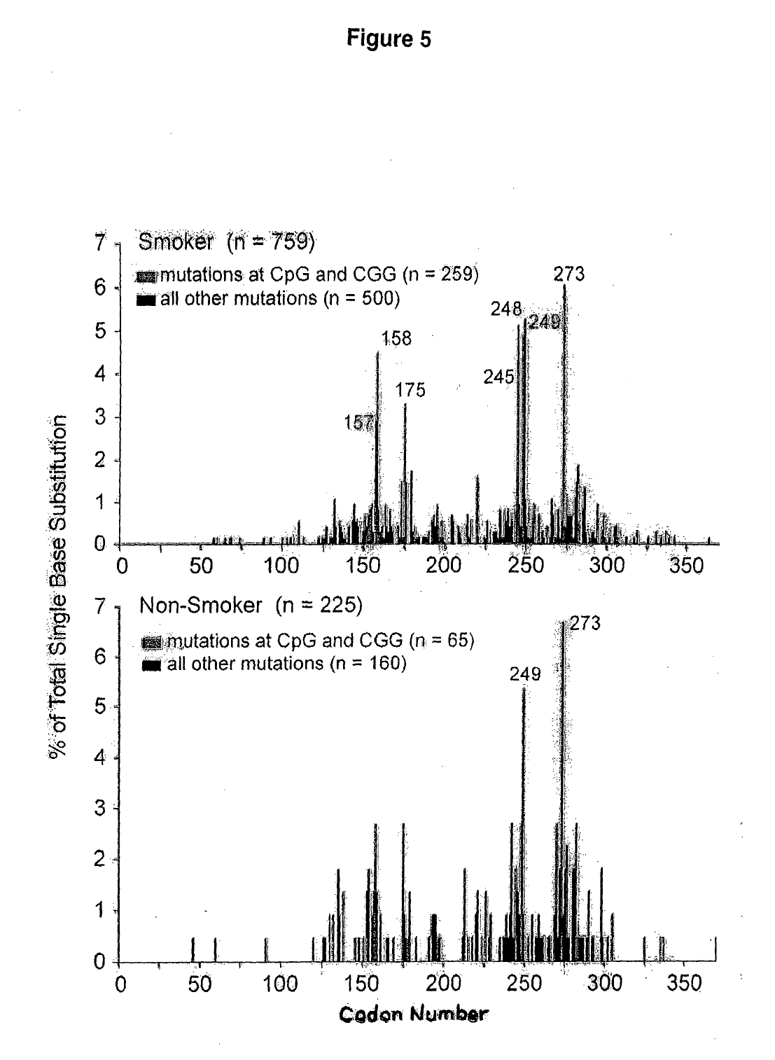

It was previously demonstrated that DNA adducts induced by diol epoxides of polycyclic aromatic hydrocarbons (PAHs), a major category of cigarette smoke (CS) carcinogens, preferentially occur at p53 mutational hotspots in CS-related lung cancers and that adducts formed at these locations are poorly repaired (Denissenko, M.-F., Pao, A., Tang, M.-s., Pfeifer, G.-P. (1996) Science 274, 430-432; Denissenko, M., Pao, A., Pfeifer, G., Tang, M.-s. (1998) Oncogene 16, 1241-1249; Smith, L.-E. Denissenko, M.-F., Bennett, W.-P., Amin, S., Tang, M.-s., Pfeifer, G.-P. (2000) JNCI 92, 803-811). The p53 gene is the most frequently mutated tumor suppressor gene in CS-related lung cancers and its mutational pattern is distinctly different from that found in lung cancers of nonsmokers (FIG. 5); PAHs have been shown to be strong carcinogens, and thus PAH-induced DNA damage may shape the p53 mutational pattern in lung cancer and may also represent a strong molecular link between lung cancer and cigarette smoking (Olivier, M., Eeles, R., Hollstein, M., Khan, M.-A., Harris, C.-C., Hainaut, P. (2002) Hum. Mutat. 19, 607-614; Greenblatt, M.-S., Bennett, W.-P., Hollstein, M., Harris, C.-C. (1994) Cancer Res. 54, 4855-4878; Denissenko, M.-F., Pao, A., Tang, M.-s., Pfeifer, G.-P. (1996) Science 274, 430-432; Denissenko, M., Pao, A., Pfeifer, G., Tang, M.-s. (1998) Oncogene 16, 1241-1249; Smith, L.-E. Denissenko, M.-F., Bennett, W.-P., Amin, S., Tang, M.-s., Pfeifer, G.-P. (2000) JNCI 92, 803-811). The question of whether PAHs are the major culprits in CS smoke that cause human cancer remains unsettled since CS contains more than 4000 compounds, many of which, particularly aldehydes, are not only more cytotoxic than PAHs but can also cause similar kinds of mutations (Hoffman, D., Hecht, S.-S. (1990) in Handbook of Experimental Pharmacology eds. Cooper, C.-S. & Grover, P.-L. (Springer-Verlag, Heidelberg), pp. 70-74; Gomes, R., Meek, M.-E., Eggleton, M. (2002) Concise International Chemical Assessment Document No. 43 World Health Organization, Geneva, Switzerland).

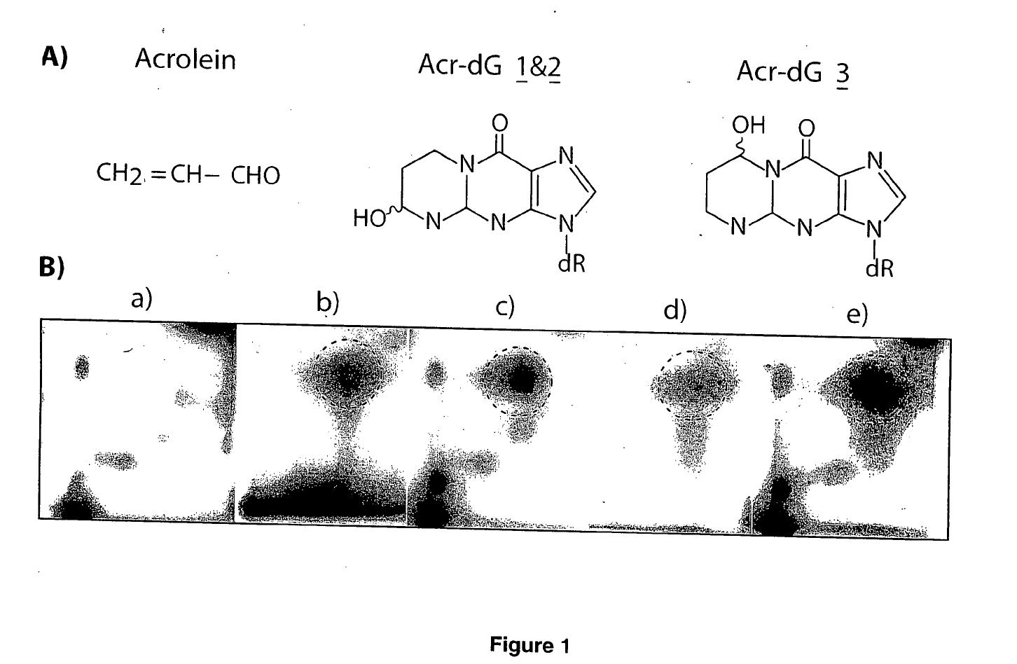

Acrolein (Acr) is one of the most abundant, reactive and mutagenic aldehydes in CS; it is found in amounts up to 1000-fold higher than those of PAHs in CS (10 to 140 μg/cigarette compared to 0.01-0.05 μg/cigarette of benzo[a]pyrene (BP)) (Hoffman, D., Hecht, S.-S. (1990) in Handbook of Experimental Pharmacology eds. Cooper, C.-S. & Grover, P.-L. (Springer-Verlag, Heidelberg), pp. 70-74). It can be taken up reasonably efficiently by human cells and react directly without metabolic activation with guanine residues in DNA to produce exocyclic DNA adducts, 6-hydroxy 1, N²-propanodexoyguanosine and 8-hydroxy 1,N²-propanodexoyguanosine adducts (Acr-dG) (FIG. 1), which are mutagenic and induce predominantly G:C to T:A transversion mutations similar to PAHs (Gomes, R., Meek, M.-E., Eggleton, M. (2002) Concise International Chemical Assessment Document No. 43 World Health Organization, Geneva, Switzerland). Although Acr has been shown to be cytotoxic and genotoxic in human cells, as a suspected carcinogen, its carcinogenicity in animal models has not been adequately evaluated due to its extremely potent toxic effects that often result in death (Gomes, R., Meek, M.-E., Eggleton, M. (2002) Concise International Chemical Assessment Document No. 43 World Health Organization, Geneva, Switzerland; Curren, R.-D., Yang. L.-L., Conklin, P.-M., Grafstrom, R.-C., Harris, C.-C. (1988) Mutat. Res. 209, 17-22; Grafstrom, R.-C., Dypbukt, J.-M., Willey, J.-C., Sundqvist, K., Edman, C., Atzori, L., Harris, C.-C. (1988) Cancer Res. 48, 1717-1721). Nonetheless, it has been found that Acr treatment greatly enhances urinary bladder papilloma occurrence in rats (Gomes, R., Meek, M.-E., Eggleton, M. (2002) Concise International Chemical Assessment Document No. 43 World Health Organization, Geneva, Switzerland). Acr is one of the two major toxic metabolites of the chemotherapeutic agents cyclophosphamide and ifosfamide, and Acr has been long suspected to be an important factor in the induction of secondary human bladder tumors in cyclophosphamide-treated patients (Gomes, R., Meek, M.-E., Eggleton, M. (2002) Concise International Chemical Assessment Document No. 43 World Health Organization, Geneva, Switzerland). Acr-dG DNA adducts have been detected in animal and human tissues (Nath, R.-G., Chung, F.-L. (1994) Proc. Nat. Acad. Sci. USA 91, 7491-7495), and it has been shown that the oral tissues of cigarette smokers have significantly higher Acr-dG levels than those of nonsmokers (Nath, R.-G., Ocando, J.-E., Guttenplan, J.-B., & Chung, F.-L. (1998) Cancer Res. 58, 581-584). Some methods of removing acrolein from gas include, but are not necessarily limited to, the use of membrane separation, catalytic oxidation, activated carbon, and silica gel containing an iron phthalocyanine catalyst. Catalytic oxidation and distillation at certain pH levels have also been used to remove acrolein from other products, including acrylonitrile. These methods include ion exchange resins and condensation, the latter procedure which has also been used to remove acrolein from acrylic acid. Other acrolein scavengers have been described (U.S. Pat. Nos. 5,760,283 and 5,606,094). These include sodium hyprochlorite, an acid salt of hydroxylamine, a urea compound, including thiourea, sodium bisulfite and 4,4-dimethyl-1-oxa-3-aza-cyclopentane. However, an important issue to consider for the use of an acrolein scavenger in a filter to remove harmful combustion products, such as that found in tobacco smoke or smoke from cooking oils, is the safety or toxicity profile of the acrolein scavenger. It would serve no purpose to combine a scavenger of acrolein with a filter unit if the acrolein scavenger poses an equal to, or greater risk than the exposure to acrolein itself. Thus, there remains a need for novel and safe scavengers of exogenous toxic molecules from combustion products, one such toxic molecule being acrolein. Furthermore, there is a need for other safe and/or non-toxic methods of removing or sequestering acrolein from tobacco smoke while retaining nicotine in tobacco.

However, it is not known whether acrolein contributes directly to CS-related mutagenesis and carcinogenesis. The present invention addresses this by utilizing the UvrABC nuclease incision method in combination with the ligation-mediated polymerase chain reaction (LMPCR) technique (UvrABC/LMPCR) to map the Acr-dG adduct distribution at the sequence level in the p. 53 gene in normal human lung cells. Moreover, the present invention also investigates whether Acr has an effect on DNA repair using host-cell reactivation (HCR) and in vitro DNA damage-specific repair synthesis assays (Feng, Z., Hu, W., & Tang, M.-s. (2004) Proc. Nat. Acad. Sci. 101, 8598-8602; Feng, Z., Hu, W., Marnett, L., & Tang, M.-s. Mutat. Res. in press).

### Exogenous Toxic Agents

The present invention relates to the identification of particular exogenous toxic agents that are present in combustion products, which upon exposure to genomic DNA or to cells, result in the formation of DNA adducts. The target for these exogenous agents appears to be the p53 gene, and more specifically, particular sites within the p53 gene, foe example, areas rich in CpG. More particularly, the target appears to be the methylated cytosines within these regions. In one particular embodiment, the exogenous toxic agent is acrolein (Acr).

The combustion products that contain numerous mutagens include smoke generated from tobacco products, as well as smoke generated from heating cooking oils. Numerous components have been identified in tobacco, which are believed to contribute to the adverse consequences of smoking. These include direct toxins, human carcinogens, mutagens, probable human carcinogens and proven animal carcinogens. Human carcinogens include benzene, 2-naphthylamine, 4-aminobiphenyl, and the radioactive element polonium-210. Probable human carcinogens include such compounds as formaldehyde, hydrazine, N-nitrosodimethylamine, N-nitrosodiethylamine, N-nitrosopyrrolidine, benzo[a]pyrene, N-nitrosodiethanolamine, and cadmium. Further compounds in tobacco smoke have been proven to be animal carcinogens, including benz[a]anthracene, butyrolactone and N-nitrosonornicotine.

In addition, it has been determined that the lung cancer incidence in Chinese women is among the highest in the world. However, further investigations determined that tobacco smoking accounts for only a minority of these cancers. On the other hand, epidemiologic investigations of lung cancer among these Chinese women have implicated exposure to indoor air pollution resulting from wok cooking, where it was determined that the volatile emissions from unrefined cooking oils are mutagenic (J. Natl. Cancer Institute (1995); 87:836-841).

The oils tested in this study included unrefined Chinese rapeseed oil, refined U.S. rapeseed (known as canola), Chinese soybean, and Chinese peanut in addition to linolenic, linoleic, and erucic fatty acids. The mutagenic potential of these heated oils was evaluated by collecting the condensates of the emissions and testing them in a Salmonella mutation assay (using Salmonella typhimurium tester strains TA98 and TA104). The volatile decomposition products were analyzed by gas chromatography and mass spectroscopy and aldehydes were detected using high-performance liquid chromatography and UV spectroscopy.

The results showed that 1,3-butadiene, benzene, acrolein, formaldehyde, and other related compounds were qualitatively and quantitatively detected, with emissions tending to be highest for unrefined Chinese rapeseed oil and lowest for peanut oil. The emission of 1,3-butadiene and benzene was approximately 22-fold and 12-fold higher, respectively, from heated unrefined Chinese rapeseed oil than from heated peanut oil.

Several other studies have implicated domestic exposure to cooking fumes as a possible risk factor, although the exact carcinogens in these studies have yet to be identified. Heterocyclic amines are known carcinogens, which have been identified in cooked meat, and also in fumes generated during frying or grilling of meats. The results of a study conducted by Seow et al. suggested that inhalation of carcinogens, such as heterocyclic amines generated during frying of meat, may increase the risk of lung cancer among smokers (Scow, A. et al. Cancer Epidemiology Biomarkers and Prevention, (2000), 9:1215-1221.

A study by Yang, et al. identified one particular compound found in the fumes from Chinese cooking oil, which was confirmed to be mutagenic. The compound was identified as 2-amino-3,8-dimethylimidazo[4,5-f]quinoxaline (MeIQx) (Yang, C. C. et al., Carcinogenesis, (1998), 19(2):359-363).

Many of the aforementioned compounds are also directly toxic to cells in the body. While the toxicologic, mutagenic and carcinogenic potential of many of these components in smoke has never been tested by direct experimentation in humans, a strong cause-and-effect relationship between smoking tobacco or inhaling the smoke from cooking oils and adverse effects has been established epidemiologically.

Although smoking of tobacco, principally cigarette smoking, but also including cigar and pipe smoking, is strongly linked epidemiologically to the aforementioned adverse sequelae, exposure to smokeless tobacco products, including chewing tobacco and snuff, also carries a risk of developing adverse health effects. Furthermore, smokers are principally exposed to what is termed “mainstream” smoke, i.e, that which is inhaled from the smoking device. However, recent studies have implicated exposure of nonsmoking individuals to what is termed “sidestream” smoke, that which arises from the smoking device itself, with adverse effects. The latter exposure has led to significant concern that individuals breathing “second-hand” smoke are at risk for developing the same adverse health consequences that typify smokers.

Methods of removing toxic components from tobacco and especially tobacco smoke, from mainstream and sidestream smoke, are desirable in reducing the excessive health care costs associated with the consequences of tobacco and tobacco smoke exposure. Furthermore, methods or devices for blocking the toxic effects of smoke generated from cooking oils to cells or tissues is proving to be of interest, in light of the studies relating the exposure of individuals to such smoke and the increase in cancer rates among these individuals.

Reduction in exposure of individuals to the toxic components in tobacco and tobacco smoke is desirable, without reducing the enjoyment of using the tobacco products. While the removal or retention of nicotine is not a feature of the instantly-claimed methods or devices, in one embodiment of the invention, retention of some or all of the nicotine content of the smoke is desired.

### Methods of Removal or Sequestering the Exogenous Toxic Agents or for Blocking the Toxic Effect on Cells or Tissues

Reduction in exposure of individuals to toxic compounds present in combustion products, particularly tobacco and tobacco smoke, as well as smoke from cooking oils, may be achieved by the agents and devices of the present invention at several points along the route either from the tobacco itself or to the point of exposure by the individual. In certain embodiments, if the toxic agent is present in the tobacco leaf, agents may be added to or blended into the tobacco itself, either smoking or smokeless tobacco, which bind and sequester toxins, not permitting them to be leached or absorbed from the smokeless tobacco or not permitting them to be volatilized into the smoke as the tobacco burns. In another embodiment, eg. for smoking tobacco, a second stage of intervention is in removing toxic products from the smoke stream. This may be achieved to some extent by toxin-sequestering agents added to the tobacco itself, which before burning act as a filter. More useful is a filter placed between the column of combusting tobacco and the mouth, or in a separate device, through which the smoke passes before entering the body. By mechanical and adsorptive properties, present filters remove particulates, tar, and other components from the smoke. At a further stage, exhaled tobacco smoke or sidestream smoke produced from the burning smoking device and present in the environment may be filtered of toxins by passing ambient room air through or in contact with a material or filter which removes toxins.

In one particular embodiment, porous, fibrous smoke filters are envisioned to remove a portion of these toxic compounds by mechanical trapping and adsorption to the fibrous surface. Nevertheless, toxic compounds remain in the inhaled smoke and contribute to enormous morbidity and mortality, mainly lung and other cancers, other lung diseases such as emphysema, and cardiovascular disease including heart attack and stroke. Numerous theories exist relating various pathophysiological disease processes with specific tobacco smoke components. It is apparent from this body of work that tobacco smoke contains toxins which are incompatible with health, and that reduction of the exposure to the body of these toxins is prudent. Except for abstaining from smoking and perhaps altering genetically the components in the tobacco leaf, reduction in exposure of the smoker to tobacco smoke toxins may be achieved only by adding toxin-sequestering agents to the tobacco or selectively removing toxins from the smoke before inhalation. On the other hand, it is desirable to avoid reducing the enjoyment of using tobacco products in accordance with the objects of the present invention.

With the identification of significant amounts of the suspected carcinogen formaldehyde in cigarette smoke, considerable effort has been expended by others on developing chemical trapping methods for removing formaldehyde from smoke, mainly by including an aldehyde-trapping chemical in the filter. This may be achieved by the inclusion of nucleophilic compounds in the filter, such as those described in the Background section above. Examples described above of filters incorporating nucleophilic compounds such as lysine apparently have not achieved their desired effect as they have not been commercially introduced.

The inventors of the present application have identified particular binding sites on the p53 gene for a particular toxic agent, acrolein. In particular, certain sequences within the gene appear to be targeted for binding by acrolein. These sequences are described in more detail below. While others have tried to remove other toxic aldehydes from smoke using filters impregnated with compounds having aldehyde reactive groups to sequester the toxic aldehydes from smoke, the success of such a filter may depend on the actual binding affinity of the toxic aldehyde for the particular reactive group on the filter. It may be that while certain aldehyde reactive groups on a chemical structure, while appearing to be sufficient for sequestering the toxic aldehydes from smoke, may not be sufficient to retain these toxic aldehydes, and they may be released from the filters during the course of smoking. On the other hand, the use of a target molecule that has the ability to sequester or remove the exogenous toxic agent and retain the binding of the toxic agent by having a greater affinity for the toxic agent, may be necessary. It was found by the inventors of the present invention, that a target gene and particular binding sites within that gene exist for one of the most prevalent toxic aldehydes in tobacco smoke, acrolein. It is also of interest to note that acrolein is also present in the smoke generated from cooking oils (see above), and may be the toxic agent responsible for the increase in cancer rates in Chinese women. Also, it is proposed that a filter designed to remove acrolein by derivatizing the filter with the particular target sequences to which acrolein binds may be a more efficient and specific means by which to remove one of the more likely candidates for the induction of mutations and carcinogenesis in humans, resulting from tobacco smoke or the inhalation of fumes from cooking oils. Thus, it is proposed that attaching either a nucleic acid or proteinaceous material to the filter, and more particularly, a nucleic acid or protein that binds with greater specificity to acrolein, may provide a more efficient means for removal of this toxic molecule from harmful combustion products.

In another particular embodiment, it is proposed that a proteinaceous material, for example, albumin, may provide a more suitable means by which to sequester toxic agents from smoke, such as acrolein, by virtue of the large number of reactive sites on the molecule. It is also readily available for simple attachment to a filter and the cost of manufacture should be much lower than that associated with attaching a chemical entity to a filter. Furthermore, another advantage to the use of albumin for attachment to a filter device is the fact that it is non-toxic, thus providing no potential threat if any of the material is released from the filter unit during smoking. U.S. Pat. No. 4,300,577 describes a filter comprising an absorptive material plus an amine-containing component which removes aldehydes and hydrogen cyanide from tobacco smoke, while U.S. Pat. No. 5,009,239 describes a filter element treated with polyethyleneimine modified with an organic acid, to remove aldehydes from tobacco smoke.

In another particular embodiment, it is proposed that attachment of the nucleic acid to a filter would serve as a good agent for trapping or sequestering an exogenous toxic agent, such as acrolein. In a more particular embodiment, it is envisioned that the nucleic acid would be selected from the regions of the p53 gene for which acrolein shows specificity. More particularly, these regions would be selected from exons 5, 7 and 8. In a more particular embodiment, the filters may be impregnated with or derivatized to contain one or more of the following target sequences from the p53 gene: the methylated CpG found at a location selected form the group consisting of codons 152, 154, 156, 157, and 158 in exon 5; codon 248 in exon 7, codons 273 and 282 in exon 8 of the p53 gene, and combinations thereof. In another particular embodiment, it is envisioned that the nucleic acid would be selected from the methylated nucleobase is found in codon 249 in exon 7 of the p53 gene. In another particular embodiment, it is envisioned that the nucleic acid would be one or more methylated CpG dinucleotides. In another particular embodiment, it is envisioned that the nucleic acid would be a methylated nucleobase, and more particularly, methylated cytosine. General methods for attaching a nucleic acid to a filter are disclosed in U.S. Pat. No. 6,117,846.

Suitable polymers to which the proteinaceous agent or nucleic acid would be attached may be selected from the group consisting of a cellulose, a starch and agarose. These polymers may be in the form of a filter unit for a cigarette, or in a face mask, or as part of an air filtration unit, or air conditioning system to remove the toxic agents from room air. Other polymers, resins or plastics of suitable porosity for use as a tobacco smoke filter or face mask or room filter or air conditioner filter are also envisioned.

Agents that may be incorporated into a filter matrix capable of trapping the toxic agents of the invention are preferably of low vapor pressure in order to remain within the filter and not become volatilized on exposure to a stream of heated air and tobacco smoke.

Suitable compounds for incorporation directly into smoking and smokeless tobacco products comprise those suitable for the intended purpose. That is, for smokeless tobacco products, suitable agents must have a toxicological profile compatible with the extent of exposure to the individual, and furthermore not interfere with the taste, flavor, or enjoyment of the product. Compounds should be of low toxicity and preferably not absorbed. For incorporation into smoking tobacco to sequester the exogenous toxic agents that form upon burning, the agents must not interfere with the flavor or enjoyment of the product, the rate of combustion of the smoking product either during or between inhalation, and not release the sequestered toxin when the agent within the tobacco is burned. The presence of the toxin-removing material should not interfere with the draw, or resistance to passage of air and smoke, through the tobacco column or filter.

### Targets of the Invention

As noted in the Examples, the inventors of the present invention have identified particular target sequences to which acrolein binds to the p53 gene, and it is proposed that due to the affinity of acrolein for these sequences, one particular strategy for removal of acrolein from combustion products is to attach any one or more of these sequences to a filter for removal of the acrolein from smoke.

In particular, acrolein appears to have particular affinity for methylated CpG dinucleotides in the p53 molecule. More particularly, the affinity appears to stem from the affinity of acrolein for methylated cytosines. The methylated cytosines are present at a location selected from the group consisting of codons 152, 154, 156, 157, and 158 of exon 5, codon 248 of exon 7, codons 273 and 282 of exon 8 of the p53 gene. It is envisioned that any one or more of the methylated CpG dinucleotides or the sequences surrounding these dinucleotides may be used for attachment to a filter unit for removal of acrolein from combustion products. A nucleic acid comprising about 2 to 100 nucleotides in length that incorporates the methylated CpG dinucleotide is suitable for attachment to a polymeric material for incorporation into a filter. More preferably, about 5 to 50 nucleotides in length is suitable for attachment to the filter, and more preferably 10 to 25 nucleotides. Suitable amounts of the nucleotides to incorporate into the filter may range from about 10 μg/cm² to about 200 μg/cm², more preferably about 20 μg/cm² to about 100 μg/cm². The methods described in U.S. Pat. No. 6,117,846, may be used for attachment of nucleic acids to a polymeric substrate for use in a filter device.

One aspect of the invention proposes to selectively disrupt or prevent acrolein present in combustion products from binding to the p53 gene in a cell by attaching the nucleic acid or protein sequences for which acrolein has demonstrated specificity to a filter unit. More particularly, in one embodiment, the attachment of these proteins or nucleic acids to a filter unit or a device comprising a filter unit to which is attached a plurality of these proteins or nucleic acid sequences, should adsorb the acrolein from smoke generated from a tobacco product or the vapor generated from cooking oils. Accordingly, it is believed that the use of these nucleic acids or proteins as described above, can significantly decrease or avoid the negative effects of acrolein on p53 function/activity. Therefore, the proteins, nucleic acids or derivatives thereof, of the invention can be used to prevent the mutagenic effect of acrolein present in combustion products on the p53 gene, thus diminishing its tumor suppressor function.

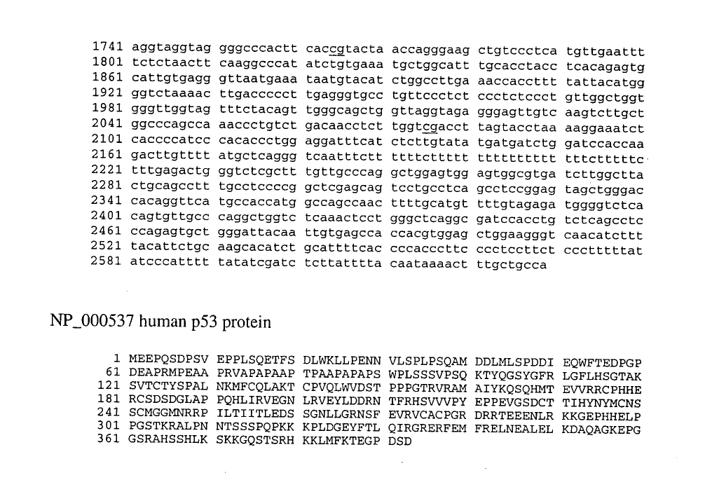

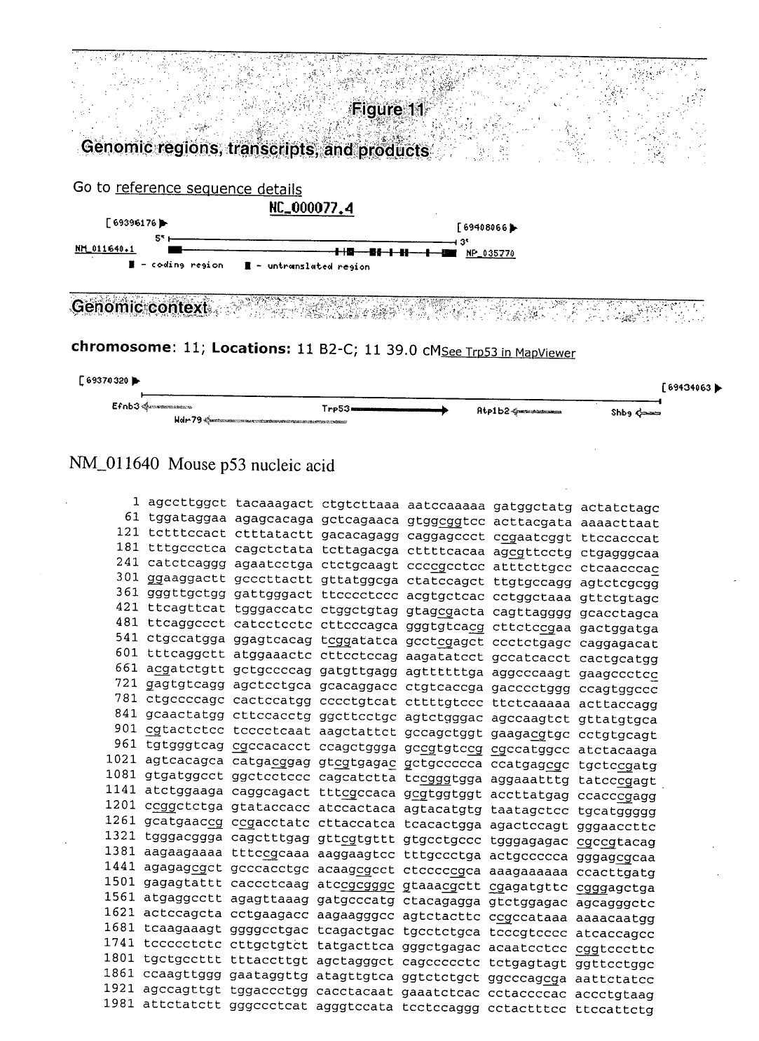

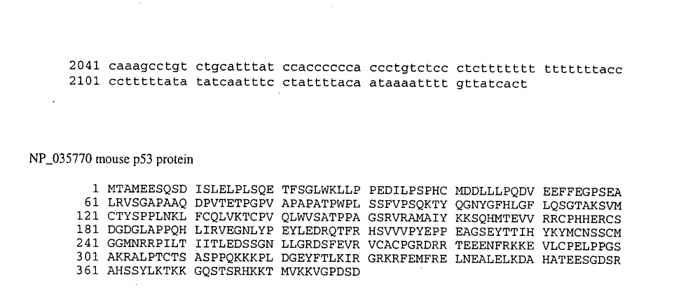

The particular target sequences that may be used for binding to a filter to remove or sequester acrolein from air or from tobacco smoke, which are obtained from the p53 gene or gene product, are outlined in FIG. 9. The sequences of particular relevance to which acrolein binds are those found in exon 5, or those found in exon 7, and those found in exon 9. Moreover, the number of nucleotides flanking the target sequences may range in size from about 1 to 20 nucleotides, or 5 to 15 nucleotides, or 10-12 nucleotides. More particularly, the target sequences to which acrolein binds in the p53 gene are those sequences containing a CpG dinucleotide, and more particularly, a methylated cytosine. The relevant portions of the p53 gene containing these binding sites are underlined in FIG. 10 (human p53) and in FIG. 11 (mouse p53). Moreover, it is anticipated that while the most cost effective means of preparing a filter to remove or sequester acrolein from a combustion product would be to chemically modify a filter, such as cellulose, to bind either a methylated cytosine or an unmethylated or methylated CpG dinucleotide, it is also contemplated that longer sequences that incorporate these moieties may be used. For example, in one embodiment, the filter for binding or sequestering acrolein from a combustion product may comprise any one or more of the underlined nucleotides in the sequence of FIG. 10 or 11, or a nucleotide sequence comprising any one of the underlined nucleotides in FIG. 10 or 11 and extending to about 1 to 20 nucleotides, or 5 to 15 nucleotides, or 10-12 nucleotides on either side of the underlined regions in the figures.

In addition, the particular target amino acid sequences that may be used for binding to a filter to remove or sequester acrolein from air or from tobacco smoke, which are encoded by the p53 gene, may be used to chemically attach to a filter or be used to impregnate a filter for the intended use. A preferred protein fragment according to the invention is a peptide encoded by those portions of the p53 gene that bind acrolein, for example, those nucleotide sequences present in exons 5, 7, or 8. Furthermore, while these fragments of the p53 gene may provide the specificity needed to bind acrolein, it is also contemplated that fragments larger than this may be effective for the intended use. Any of the amino acids in these sequences may be substituted with a conservative amino acid, and still retain their ability to bind to acrolein. Also included within the scope of the provided fragments are peptides of wherein one or more, preferably one, two or three amino acid residues are replaced with another natural or unnatural amino acids.

A derivative of any of the fragments noted above may be used to bind to filters in order to remove or sequester acrolein, according to the invention. Such derivatives may involve one or multiple modifications as compared to a peptide of the invention, e.g. carry one or more of the above defined moieties. In other words, a derivative of the invention is intended to include compounds derivable from or based on a peptide of the invention or another derivative of the invention. The preferred derivatives of the invention are capable of binding to acrolein and of selectively inhibiting or blocking the binding of acrolein to the p53 gene or gene product (protein). The ability of a test compound to inhibit the interaction between acrolein and p53 can be shown by assays commonly known in the art, or modifications of known assays readily apparent to a person of ordinary skill in the art. Suitable assays include e.g. a binding assay determining binding of a test compound, e.g. a compound of the invention, to acrolein an in vitro assay. Assays may be performed qualitatively or quantitatively and require comparison to one or more suitable controls. A preferred binding assay is a competitive binding assay. The principle underlying a competitive binding assay is generally known in the art. Briefly, such binding assay is performed by allowing a compound to be tested for its capability to compete with a known, suitably labeled ligand, e.g. acrolein or p53 for the binding site at a target molecule, e.g. p53 or acrolein (depending on which molecule is used as known ligand). A suitably labeled ligand is e.g. a radioactively labeled ligand or a ligand which can be detected by its optical properties, such as absorbance or fluorescence. After removing unbound ligand and test compound the amount of labeled ligand bound to the target protein is measured. If the amount of bound ligand is reduced in the presence of the test compound, said compound is found to bind to the target molecule. For example, ELISA-type assays may be used wherein p53 or an appropriately labeled p53 peptide comprising the acrolein binding site on p53 is immobilized and binding of acrolein is competed for by a candidate inhibitor. Alternatively, acrolein may be immobilized and binding of p53 is competed for by such candidate. Furthermore, an assay involving phage display of a candidate peptide, e.g. a phage ELISA assay, may be used.

The peptides and derivatives of the present invention can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof. Likewise, the methods for synthesizing a nucleotide of the invention are also known to those skilled in the art.

As noted above, and according to another embodiment of the invention, the acrolein contained in combustion products may be removed by filtering the air containing the combustion products through filters to which has been coupled or which has been impregnated with, any of the above noted sequences. This can be accomplished, among other ways, by employing the DNA fragments or protein or peptide fragments, for attachment to an insoluble polymeric support such as agarose, cellulose and the like. After binding, all non-binding components can be washed away, leaving acrolein bound to the DNA/solid support, or protein/solid support. The acrolein can be quantitated by any means known in the art. It can be determined using mass spectrometry or HPLC.

In accordance with the present invention, in order to make the solid support, for example, a cellulose filter, chemically receptive to various additives, the cellulose is modified. Procedures for modifying cellulose in preparation for binding a compound such as acrolein are well known to those skilled in the art. For instance, the cellulose may be modified through a surface modification process that forms chemical moieties on the surface of the cellulose. A chemical additive is then selected that contains a functional moiety that reacts with the moieties on the surface of the cellulose. Thus, a chemical linkage, such as a covalent bond, is formed between the chemical additive and the cellulose material. Because the chemical additive is chemically bonded to the cellulose material, retention of the chemical additive on the cellulose is dramatically improved.

The moiety that is formed on the surface of the cellulose can vary depending upon the particular application. In one embodiment, for instance, the cellulose can be modified to form a moiety that comprises an aldehyde reactive group.

Once the cellulose is modified to contain a moiety as described above and contacted with a chemical additive containing a corresponding moiety, a chemical reaction occurs forming a chemical linkage between the cellulose and the chemical additive. For many applications, for instance, a covalent bond forms between the modified cellulose and the chemical additive. In other embodiments, however, other bonds may form including other physiochemical bonds, hydrogen bonds, and the like. The bonding mechanism, however, must be sufficiently strong so as the attachment of the chemical additive to the fibers survives dilution forces and shear forces present in the processes used to manufacture the articles comprising the modified cellulose and chemical additive.

### Nucleic Acids of the Present Invention

The invention provides for the use of nucleic acids derived from or obtained from the p53 gene, or homologs thereof, and fragments or portions thereof. Preferred nucleic acids have a sequence at least about 50%, 60%, 65%, 70%, 75%, 80%, and more preferably 85% homologous and more preferably 90% and more preferably 95% and even more preferably at least 99% homologous with a nucleotide sequence of a subject gene, e.g., a p53 gene. Nucleic acids at least 90%, more preferably 95%, and most preferably at least about 98-99% identical with a nucleic sequence represented in one of the subject nucleic acids of the invention or complement thereof are of course also within the scope of the invention. In preferred embodiments, the nucleic acid is mammalian and in particularly preferred embodiments, includes all or a portion of the nucleotide sequence corresponding to the coding or promoter region, which correspond to the coding sequences or promoter sequences of the subject p53 gene or homolog or fragment-encoding DNAs. In a particular embodiment, the nucleic acid encoding the p53 gene or fragments thereof is the human p53 sequence as set forth in SEQ ID NO: 1. (GenBank Accession numbers 7157; NM_{—}000546 and NP_{—}000537). In another particular embodiment, the nucleic acid encoding the p53 gene or fragments thereof is the mouse p53 sequence as set forth in SEQ ID NO: 3. (GenBank Accession numbers NC_{—}000077.4; NM_{—}011640.1 and NP_{—}035770).

The invention also pertains to isolated nucleic acids comprising a nucleotide sequence encoding p53 polypeptides, variants and/or equivalents of such nucleic acids. The term equivalent is understood to include nucleotide sequences encoding functionally equivalent p53 polypeptides or functionally equivalent peptides having an activity of a p53 protein such as described herein. Equivalent nucleotide sequences will include sequences that differ by one or more nucleotide substitution, addition or deletion, such as allelic variants; and will, therefore, include sequences that differ from the nucleotide sequences of e.g. the corresponding p53 gene GenBank entries due to the degeneracy of the genetic code.

Preferred nucleic acids are vertebrate p53 nucleic acids. Particularly preferred vertebrate p53 nucleic acids are mammalian. Regardless of species, particularly preferred p53 nucleic acids encode polypeptides that are at least 50%, 60%, 65%, 70%, 75%%, 80%, 85%, 90%, 95%, or 99% similar or identical to an amino acid sequence of a vertebrate p53 protein. In one embodiment, the nucleic acid is a cDNA encoding a polypeptide having at least one bio-activity of the subject p53 polypeptides. Preferably, the nucleic acid includes all or a portion of the nucleotide sequence corresponding to the nucleic acids available through GenBank.

Still other preferred nucleic acids of the present invention encode a p53-encoding polypeptide which is comprised of at least 2, 5, 10, 25, 50, 100, 150 or 200 amino acid residues. For example, such nucleic acids can comprise about 50, 60, 70, 80, 90, or 100 base pairs. Also within the scope of the invention are nucleic acid molecules for use as probes/primer or antisense molecules (i.e. noncoding nucleic acid molecules), which can comprise at least about 6, 12, 20, 30, 50, 60, 70, 80, 90 or 100 base pairs in length. Such probes may be used for the screening of candidate compounds that may be used in the manner of the present invention for preventing the mutagenic or toxic effects of acrolein or other exogenous toxic agents in tobacco smoke or smoke from cooking oils.

Another aspect of the invention provides a nucleic acid which hybridizes under stringent conditions to a nucleic acid represented by any of the subject nucleic acids of the invention. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6 or in Molecular Cloning: A Laboratory Manual, Cold Spring Harbor Press (1989). For example, the salt concentration in the wash step can be selected from a low stringency of about 2.0×. SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature and salt concentration may be held constant while the other variable is changed. In a preferred embodiment, an antigen nucleic acid of the present invention will bind to one of the subject SEQ ID NOs. or complement thereof under moderately stringent conditions, for example at about 2.0×. SSC and about 40° C. In a particularly preferred embodiment, a p53 encoding nucleic acid of the present invention will bind to one of the nucleic acid sequences of SEQ ID NO: 1 or SEQ ID NO: 3 or a complement thereof under high stringency conditions. In another particularly preferred embodiment, a p53-encoding nucleic acid sequence of the present invention will bind to one of the nucleic acids of the invention which correspond to a p53-encoding ORF nucleic acid sequences, under high stringency conditions.

Nucleic acids having a sequence that differs from the nucleotide sequences shown in one of the nucleic acids of the invention or complement thereof due to degeneracy in the genetic code are also within the scope of the invention. Such nucleic acids encode functionally equivalent p53 peptides (i.e., peptides having a biological activity of a p53-encoding polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a p53 polypeptide. However, it is expected that DNA sequence polymorphisms that do lead to changes in the amino acid sequences of the subject p53 polypeptides will exist among mammals. One skilled in the art will appreciate that these variations in one or more nucleotides (e.g., up to about 3-5% of the nucleotides) of the nucleic acids encoding polypeptides having an activity of a p53-encoding polypeptide may exist among individuals of a given species due to natural allelic variation.

### Screening for Novel Candidate Compounds and Agents

“Candidate compound” or “test compound” refers to any compound or molecule that is to be tested, and more particularly for the present invention, for its ability to inhibit the binding of a mutagenic agent to p53. As used herein, the terms, which are used interchangeably, refer to biological or chemical compounds such as simple or complex organic or inorganic molecules, peptides, proteins, peptidomimetics, peptide mimics, antibodies, nucleic acids (DNA or RNA), including oligonucleotides, polynucleotides, dinucleotides, nucleobases, antisense molecules, small interfering nucleic acid molecules, such as siRNA or shRNA molecules, carbohydrates, lipoproteins, lipids, small molecules and other drugs. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Agents or candidate compounds can be randomly selected or rationally selected or designed. As used herein, an agent or candidate compound is said to be “randomly selected” when the agent is chosen randomly without considering the specific interaction between the agent and the target compound or site. As used herein, an agent is said to be “rationally selected or designed”, when the agent is chosen on a nonrandom basis which takes into account the specific interaction between the agent and the target site and/or the conformation in connection with the agent's action. Moreover, the agent may be selected by its effect on the gene expression profile obtained from screening in vitro or in vivo. For example, the gene expression data for activated or suppressed macrophages or monocytes (including Kupffer cells) can be accessed online through databases including Pub Med, Human Genome Project (HGP), Gene Bank and PDB (Protein Data Bank). Furthermore, candidate compounds can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91: 11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

If the screening for compounds is done with a library of compounds, it may be necessary to perform additional tests to positively identify a compound that satisfies all required conditions of the screening process. There are multiple ways to determine the identity of the compound. One process involves mass spectrometry, for which various methods are available and known to the skilled artisan (see for instance neogenesis.com).

Any screening technique known in the art can be used to screen for agents that remove or sequester exogenous toxic agents from combustion products, or for the ability to block the toxic or mutagenic effect of agents like acrolein on cells or tissue. The present invention contemplates screens for agents that bind or sequester acrolein, as well as screens for agents that protect a cell or tissue against the toxic or mutagenic effect of acrolein in vitro or in vivo. For example, natural products or peptide libraries can be screened using assays of the invention for molecules that have the ability to bind acrolein or to block the formation of DNA adducts in a cell, or tissue, or isolated DNA. Methods for measuring adduct formation are known to those skilled in the art. In one particular embodiment, the inventors have utilized a method comprised of the UvrABC-nuclease incision method, in combination with the ligation-mediated polymerase chain reaction to measure acrolein-DNA adducts (See the example section). If a candidate compound or test compound is incubated with a cell or cell line in vitro that contains the p53 gene, and it proves to prevent DNA adduct formation within the p53 gene, it may then be further screened in vivo in an animal model for the ability to prevent tumor formation after exposure of the animal to a mutagenic or carcinogenic agent.

Another approach uses recombinant bacteriophage to produce large libraries. Using the “phage method” [Scott and Smith, 1990, Science 249:386-390 (1990); Cwirla, et al., Proc. Natl. Acad. Sci., 87:6378-6382 (1990); Devlin et al., Science, 249:404-406 (1990)], very large libraries can be constructed (10⁶-10⁸ chemical entities). A second approach uses primarily chemical methods, of which the Geysen method [Geysen et al., Molecular Immunology 23:709-715 (1986); Geysen et al. J. Immunologic Method 102:259-274 (1987)] and the method of Fodor et al. [Science 251:767-773 (1991)] are examples. Furka et al. [14th International Congress of Biochemistry, Volume 5, Abstract FR:013 (1988); Furka, Int. J. Peptide Protein Res. 37:487-493 (1991)], Houghton [U.S. Pat. No. 4,631,211, issued December 1986] and Rutter et al. [U.S. Pat. No. 5,010,175, issued Apr. 23, 1991] describe methods to produce a mixture of peptides that can be tested by the methods described above.

Screening phage-displayed random peptide libraries offers a rich source of molecular diversity and represents a powerful means of identifying peptides that bind a molecule of interest (Cwirla, et al., Proc. Natl. Acad. Sci., 87:6378-6382 (1990); Devlin et al., Science, 249:404-406 (1990)). Phage expressing binding peptides are selected by affinity purification with the target of interest. This sytem allows a large number of phage to be screened at one time. Since each infectious phage encodes the random sequence expressed on its surface, a particular phage, when recovered from an affinity matrix, can be amplified by another round of infection. Thus, selector molecules immobilized on a solid support can be used to select peptides that bind to them. This procedure reveals a number of peptides that bind to the selector and that often display a common consensus amino acid sequence. Biological amplification of selected library members and sequencing allows the determination of the primary structure of the peptide(s).

Peptides are expressed on the tip of the filamentous phage M13, as a fusion protein with the phage surface protein pilus (at the N-terminus). Typically, a filamentous phage carries on its surface 3 to 5 copies of pili and therefore of the peptide. In such a system, no structural constraints are imposed on the N-terminus; the peptide is therefore free to adopt many different conformations, allowing for a large diversity. However, biases in the distribution of peptides in the library may be caused by biological selection against certain of the peptides, which could reduce the diversity of peptides contained in the library. In practice, this does not appear to be a significant problem. When randomly selected peptides expressed at the N-terminus of pili were analyzed (Cwirla, et al., Proc. Natl. Acad. Sci., 87:6378-6382 (1990)), most amino acids appeared at each position of the variable peptide, indicating that no severe discrimination against particular amino acids had occurred. Selection against particular combinations of amino acids would however not have been detected in this analysis.

In another aspect, synthetic libraries [Needels et al., Proc. Natl. Acad. Sci. USA 90: 10700-4 (1993); Ohlmeyer et al., Proc. Natl. Acad. Sci. USA 90:10922-10926 (1993); Lam et al., international Patent Publication No. WO 92/00252; Kocis et al., International Patent Publication No. WO 9428028, each of which is incorporated herein by reference in its entirety], and the like can be used to screen for novel peptides or mimics thereof or fragments thereof according to the present invention.

Alternatively, the effect of a candidate compound may be tested for the ability to prevent apoptosis induced by a carcinogenic or mutagenic agent. Methods for measuring apoptosis are well known to those skilled in the art. For example, such methods include, but are not limited to annexin V staining, DNA laddering, staining with dUTP and terminal transferase [TUNEL].

In a more particular embodiment, the method of screening for a candidate compound that prevents the binding of a p53 tumor suppressor inhibitor to a p53 molecule, wherein the binding results in abrogation of the tumor suppressing activity or function of the p53 molecule, comprises:

(a) contacting the p53 molecule, or fragments thereof, or genomic DNA comprising the p53 molecule, with a candidate compound in the presence or absence of a known inhibitor, wherein said p53 molecule has the nucleic acid sequence of SEQ ID NO: 1 or SEQ ID NO: 3 and/or the amino acid sequence of SEQ ID NO: 2 or SEQ ID NO: 4; and

(b) determining the level of p53 expression or activity/function in the presence or absence of the candidate compound;

wherein the candidate compound is considered to be effective if the level of p53 expression or activity/function is higher in the presence of the candidate compound as compared to in the absence of the candidate compound.

The methods used to measure the effect of the candidate compound on p53 expression may include standard procedures known to those skilled in the art. For example, the level of expression of a gene or gene product (protein) may be determined by a method selected from, but not limited to, cDNA microarray, reverse transcription-polymerase chain reaction (RT-PCR), real time PCR and proteomics analysis. Other means such as electrophoretic gel analysis, enzyme immunoassays (ELISA assays), Western blots, dotblot analysis, Northern blot analysis and in situ hybridization may also be contemplated for use, although it is to be understood that the former assays that are noted (eg. micrarrays, RT-PCR, real time PCR and proteomics analysis) provide a more sensitive, quantitative and reliable measurement of genes or gene products that are modulated by a candidate CSP or an analogue, mimic or fragment thereof. Sequences of the genes or cDNA from which probes are made (if needed) for analysis may be obtained, e.g., from GenBank.

The method may further comprise treating a tumor bearing animal with a candidate compound capable of increasing the level of expression or function of the p53 molecule, and assessing the effect of the candidate compound on the growth, progression or metastasis of the tumor,

wherein the tumor arises as a result of loss of function of the p53 molecule or wherein the p53 molecule is mutated as a result of exposure of a cell in the animal to a mutagen;

and wherein a candidate compound effective at inhibiting the growth, progression, or metastasis of a tumor in the animal is identified as a positive candidate compound.

In yet another embodiment, a method of screening for a candidate compound capable of inhibiting the binding of a mutagenic agent to a p53 molecule, or to genomic DNA containing a p53 molecule, or to a fragment, nucleobase or dinucleotide derived therefrom, comprises:
(a) contacting the p53 molecule, or a fragment, nucleobase or dinucleotide derived therefrom, or genomic DNA containing the p53 gene, with a known mutagenic agent in the absence and presence of a candidate compound, wherein the p53 molecule is:
   (i) a DNA corresponding to SEQ ID NO: 1 or SEQ ID NO: 3, and wherein the nucleic acid fragment, nucleobase, or dinucleotide is obtained from exons 5, 7 or 8 of the p53 gene of SEQ ID NO: 1 or SEQ ID NO: 3;
   (ii) a protein comprising SEQ ID NO: 2 or SEQ ID NO: 4, or a fragment derived therefrom;
   (v) a nucleic acid comprising a sequence hybridizable to SEQ ID NO: 1 or SEQ ID NO: 3 or a complement thereof under conditions of high stringency, or a protein comprising a sequence encoded by said hybridizable sequence; or
   (vi) a nucleic acid at least 90% homologous to SEQ ID NO: 1 or SEQ ID NO: 3 or a complement thereof as determined using an NBLAST algorithm or a protein encoded thereby;
(d) determining whether or not the candidate compound blocks the mutagenic effect of the known mutagenic agent on the p53 molecule.

The effect of a known mutagenic agent on the p53 molecule may be measured by assessing the formation of one or more DNA adducts in the p53 gene or a fragment thereof. Adduct formation may be measured by any means known to those skilled in the art. One particular means of measuring such adduct formation is by the UVR-BC/LMPCR method, as described herein.

This method may further comprise treating a tumor bearing animal with the candidate compound so identified and assessing the effect of the candidate compound on the growth, progression or metastasis of the tumor, wherein said tumor arises as a result of loss of function of the p53 molecule or wherein the p53 molecule is mutated as a result of exposure of a cell in said animal to a mutagen; wherein a candidate compound effective at inhibiting the growth, progression, or metastasis of a tumor in said animal is identified as a positive candidate compound.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Acrolein as a Major Mutagenic or Carcinogen Agent: Binding at p53 Mutational Hotspots and Inhibition of DNA Repair

### Materials and Methods

Cell Culture, Carcinogen Treatment, and Genomic DNA Isolation. Normal human bronchial epithelial (NHBE) cells were cultured in medium provided by Clontics (San Diego, Calif.). Normal human lung fibroblasts (NHLF) (CCL-202) and lung adenocarcinoma cells (A549) (American Type Culture Collection, Manassas, Va.) were grown in MEM medium supplemented with 10% fetal bovine serum. Stock solutions of Acr (Sigma-Aldrich, St. Louis, Mo.) and BPDE (Chemsyn Science Laboratories, Lenexa, Kans.) were prepared immediately before use. Cells at 70% confluency were washed with phosphate buffer (PBS, 70 mM NaCl, 2 mM KCl, 1 mM KH₂PO₄, pH 7.4) and treated with different concentrations of Acr (0-100 μM) in serum-free culture medium for 6 h, or different concentrations of BPDE for 30 min, at 37° C. in the dark. After treatment, the genomic DNA was isolated as previously described (Denissenko, M.-F., Pao, A., Tang, M.-s., Pfeifer, G.-P. (1996) Science 274, 430-432; Denissenko, M., Pao, A., Pfeifer, G., Tang, M.-s. (1998) Oncogene 16, 1241-1249; Smith, L.-E. Denissenko, M.-F., Bennett, W.-P., Amin, S., Tang, M.-s., Pfeifer, G.-P. (2000) JNCI 92, 803-811). For in vitro modifications, genomic DNA was isolated from untreated cells and dissolved in H₂O, mixed with different concentrations of Acr, and incubated at 37° C. for 12 h. After repeated phenol and diethyl ether extractions, the DNA was then precipitated with ethanol and dissolved in TE (10 mM Tris, pH 7.5, 1 mM EDTA) buffer.

Acr-DNA Adduct Analysis. Acr-DNA adducts formed in cells treated with Acr (0-100 μM) and in purified genomic DNA modified with Acr (0-100 μM) were analyzed by the ³²P post-labeling and two dimensional thin layer chromatography (2D TLC) method on polyethyleneimine-cellulose sheets (Anatech, Newark, Del.), as described by Eder and Budiawan (Eder, E. & Budiawan (2001) Cancer Epidemiol. 10, 883-888). The solvents used are: D1, 0.7 M ammonium formate (pH 3.5); D2, 0.3 M ammonium sulfate (pH 7.5). The chromatograms were visualized by autoradiography, the Acr-DNA adducts (although all ³²P labeled Acr-dG adducts are in 3′,5′-bisphosphate forms, for the sake of simplicity remain labeled as Acr-dG adducts) were excised and the radioactivity was measured. Acr-DNA adducts levels were calculated by determining the relative adduct labeling, which is the ratio of labeled adduct nucleotides to labeled total nucleotides. The specific radioactivity of [γ-³²P] ATP, determined by labeling a known amount of dGMP (deoxyadenosine 3′-phosphate) (Sigma), was used for the relative labeling calculations. The well characterized Acr-dG 2 and Acr-dG 3 adducts and oligomer containing Acr-dG 3 kindly provided by Dr. Fung-Lung Chung, Georgetown University, and Drs. Carmelo Rizzo and Larry Marnett, respectively, were used as standards (Chung, F.-L., Young, R., & Hecht, S.-S. (1984) Cancer Res. 44, 990-995).

Preparation of ³²P-end Labeled p53 DNA Fragments and 5-C Cytosine Methylation at CpG Sites. DNA fragments of 247 by 5′-³²P end-labeled p53 exon 5 and 141 by 3′-³²P-end-labeled p. 53 exon 7 were prepared according to the previously described method (Feng, Z., Hu, W., Rom, W.-N., Beland, F.-A., Tang, M.-s. (2002) Biochemistry 41, 6414-6421). These ³²P-end-labeled p53 DNA fragments were subjected to SssI methylase (New England Biolabs, Beverly, Mass.) treatment in the presence of S-adenosylmethionine (SAM) according to manufacturer's instructions to methylate all cytosines at CpG sites.

Acr Modification of Supercoiled Plasmids and DNA Fragments and UvrABC Incision Assay of Acr-dG Adducts. Supercoiled pGEM plasmids, purified as described previously (Feng, Z., Hu, W., & Tang, M.-s. (2004) Proc. Nat. Acad. Sci. 101, 8598-8602), and ³²P-labeled DNA fragments were modified with different concentrations of Acr solution and purified as described above; after ethanol precipitation DNA was dissolved in TE buffer. Methods for UvrA, UvrB, and UvrC proteins purifications, UvrABC nuclease incision assays, separations of the resultant DNA, and quantification of band intensity were the same as previous described (Denissenko, M.-F., Pao, A., Tang, M.-s., Pfeifer, G.-P. (1996) Science 274, 430-432; Denissenko, M., Pao, A., Pfeifer, G., Tang, M.-s. (1998) Oncogene 16, 1241-1249; Smith, L.-E. Denissenko, M.-F., Bennett, W.-P., Amin, S., Tang, M.-s., Pfeifer, G.-P. (2000) JNCI 92, 803-811, Tang, M.-s. (1996) in Technologies for Detection of DNA Damage and Mutation ed. Pfeifer, G. (Plenum Press, New York) pp. 139-152).

Mapping DNA Adduct Distribution in the p53 Gene in Human Genomic DNA Using the UvrABC/LMPCR Method. UvrABC/LMPCR method was the same as described previously (Denissenko, M.-F., Pao, A., Tang, M.-s., Pfeifer, G.-P. (1996) Science 274, 430-432; Feng, Z., Hu, W., Chen, J.-X., Pao, A., Li, H., Rom, W., Hung, M.-C., Tang, M.-S. (2002) J. Natl. Cancer Inst. 94, 1527-1536). Each experiment was repeated 3 times, with very similar results. The calculation of the relative intensities of DNA adduct formation at different codons in the p5.3 gene was performed in the same manner as previously described (Denissenko, M.-F., Pao, A., Tang, M.-s., Pfeifer, G.-P. (1996) Science 274, 430-432; Feng, Z., Hu, W., Chen, J.-X., Pao, A., Li, H., Rom, W., Hung, M.-C., Tang, M.-S. (2002) J. Natl. Cancer Inst. 94, 1527-1536). We used various concentrations of Acr for treating cells in this study, and found that concentrations did not qualitatively affect the Acr-DNA binding patterns. Thus, for the sake of clarity, results from one concentration were used for the quantitative determinations.

Determination of the Effect of Acr Treatment on DNA Repair. Host cell reactivation (HCR) and in vitro DNA repair synthesis assays were performed as previously described to determine the effects of Acr treatment on DNA repair in human cells (Feng, Z., Hu, W., & Tang, M.-s. (2004) Proc. Nat. Acad. Sci. 101, 8598-8602; Feng, Z., Hu, W., Marnett, L., & Tang, M.-s. Mutat. Res. in press).

Determination of Mutations Induced by Acr-DNA Adducts. The methods used for mutation detection and for mutational spectrum determination were the same as previously described (Feng, Z., Hu, W., Amin, S., & Tang, M.-s. (2003) Biochemistry 42, 7848-7854). Briefly, shuttle vector pSP189 plasmid DNA was modified with different concentrations of Acr, and transfected into NHLF for replication. Plasmids were recovered 72 h after transfection, and replicated plasmids were then transformed into MB7070 E. coli indicator cells. The supF gene in plasmids isolated from mutant white colonies were then sequenced.

### Results

Acr Can Directly Modify DNA to Form Propanodeoxyguanine Adducts in vitro and in vivo. It is well known that Acr can react directly with guanine residues in purified DNA to produce four isomeric exocyclic DNA adducts, two minor stereoisomeric 6-hydroxy 1, N²-propanodexoyguaosine adducts (Acr-dG 1 and 2) and two major stereoisomeric 8-hydroxy 1,N²-propanodexoyguaosine adduct (Acr-dG 3 and 4) (Chung, F.-L., Young, R., & Hecht, S.-S. (1984) Cancer Res. 44, 990-995; F.L. Chung and C. Rizzo, personal communication) (FIG. 1). To ensure that the Acr-DNA adducts formed in vivo resulted from the direct interaction of Acr with genomic DNA, we identified the type of DNA adducts formed in cultured lung cells treated with Acr and in purified genomic DNA treated with Acr using the ³²P post-labeling and two dimensional TLC method. The results in FIG. 1 show that Acr-dG 3 is the major Acr-dG adduct found in both Acr-treated purified genomic DNA and in DNA from cells treated with Acr. Three isomeric Acr-dG adducts are found in Acr modified dGMP, with the major adduct (Acr-dG 3) being the same as found in Acr treated cells and genomic DNA. No Acr-dG 1 or Acr-dG 2 adducts were found in Acr-treated cells of Acr-modified genomic DNA. While a small quantity of Acr-dG 4 adducts were found in Acr-treated cells, this type of adducts was not found in Acr-modified genomic DNA. Using relatively high concentrations of Acr (1.1 M) for modifications of calf thymus DNA, Chung et al. detected Acr-dG 1 and 2 adducts (Chung, F.-L., Young, R., & Hecht, S.-S. (1984) Cancer Res. 44, 990-995). These two types of adducts were not evident in Acr cultured Chinese hamster ovary cells, liver samples from mice and rats, and human liver and oral tissue samples; in contrast, Acr-dG 3 adducts were detected in these samples (Nath, R.-G., Chung, F.-L. (1994) Proc. Nat. Acad. Sci. USA 91, 7491-7495; Nath, R.-G., Ocando, J.-E., Guttenplan, J.-B., & Chung, F.-L. (1998) Cancer Res. 58, 581-584; Foiles, P.-G., Akerkar, S.-A., Miglietta, L.-M. and Chung, F.-L. (1990) Carcinogenesis 11, 2059-2061). Structural constraints in genomic DNA and chromatin structure may hinder formation of Acr-dG 1 and 2 adduct isomers. Our results are consistent with well-established findings that Acr can react directly without metabolic activation with guanine residues in DNA to produce exocyclic propanodeoxyguanosine DNA adducts (Nath, R.-G., Chung, F.-L. (1994) Proc. Nat. Acad. Sci. USA 91, 7491-7495; Nath, R.-G., Ocando, J.-E., Guttenplan, J.-B., & Chung, F.-L. (1998) Cancer Res. 58, 581-584, Chung, F.-L., Young, R., & Hecht, S.-S. (1984) Cancer Res. 44, 990-995; Foiles, P.-G., Akerkar, S.-A., Miglietta, L.-M. and Chung, F.-L. (1990) Carcinogenesis 11, 2059-2061; Nath, R.-G., Chen, H.-J.-C., Nishikawa, A., Young-Sciame, R. & Chung, F.-L. (1994) Carcinogenesis 15, 979-984).

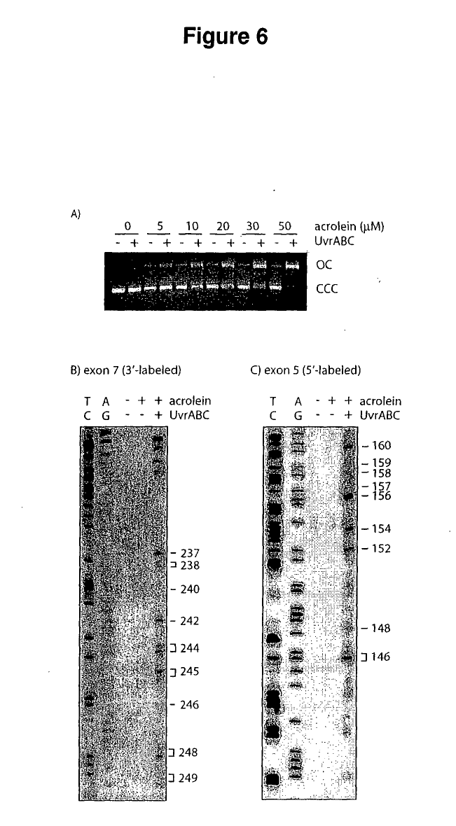

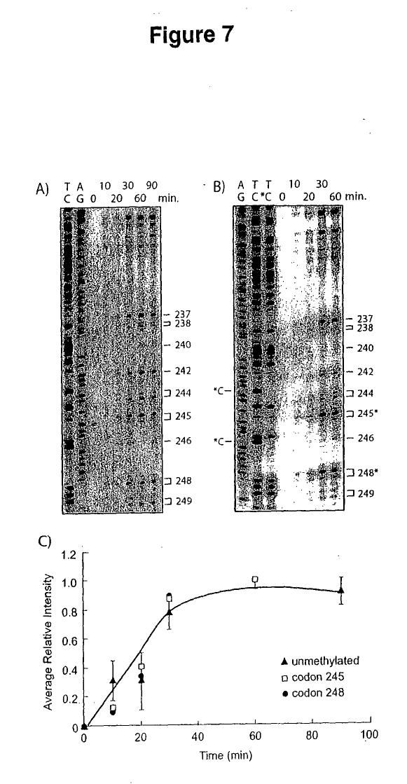

UvrABC Is Able to Incise Acr-dG Adducts Quantitatively and Specifically. In order to assess the contribution of Acr-induced DNA damage to the p53 mutation pattern in lung cancer it is necessary to map the Acr-DNA adduct distribution at the sequence level in the p53 gene of lung cells treated with Acr. Previously, using the UvrABC/LMPCR method, we successfully mapped various types of bulky carcinogen-induced DNA damage at the sequence level in the p53 and ras genes (Denissenko, M.-F., Pao, A., Tang, M.-s., Pfeifer, G.-P. (1996) Science 274, 430-432; Feng, Z., Hu, W., Rom, W.-N., Beland, F.-A., Tang, M.-s. (2002) Biochemistry 41, 6414-6421; Feng, Z., Hu, W., Chen, J.-X., Pao, A., Li, H., Rom, W., Hung, M.-C., Tang, M.-S. (2002) J. Natl. Cancer Inst. 94, 1527-1536; Feng, Z., Hu, W., Rom, W.-N., Beland, F.-A., & Tang, M. s. (2002) Carcinogenesis 23, 1721-1727). The rationale of this approach is based on the finding that, under proper conditions, UvrABC can incise bulky DNA adducts specifically and quantitatively, and the extent of UvrABC incision therefore represents the extent of adduct formation rather than UvrABC sequence preferences (Tang, M.-s. (1996) in Technologies for Detection of DNA Damage and Mutation ed. Pfeifer, G. (Plenum Press, New York) pp. 139-152). Since radioactively labeled Acr is not available, we assessed the quantitative relationship between UvrABC incision and Acr-DNA adduct formation in supercoiled DNA. We found that the number of UvrABC incisions was proportional to the concentration of Acr used for DNA modification, indicating that UvrABC is able to cut Acr-dG adducts quantitatively (FIG. 6A). To determine the specificity of UvrABC cutting, 5′ or 3′ single-end, ³²P-labeled p. 53 DNA fragments were reacted with UvrABC nuclease and the resultant DNAs were separated by electrophoresis in a denatured DNA sequencing gel. The results show that UvrABC makes the typical dual incisions 7 nucleotides 5′ and 4 nucleotides 3′ to an Acr-dG adduct, similar to what we have found for most bulky carcinogen-induced DNA damage (Tang, M.-s. (1996) in Technologies for Detection of DNA Damage and Mutation ed. Pfeifer, G. (Plenum Press, New York) pp. 139-152) (FIGS. 6B&C). The results also show that the kinetics of UvrABC cutting at different sequences are similar, if not identical (FIGS. 7A&C). It should be noted that we have found that at the end of incubation, UvrABC remains active. Based on these results we concluded that UvrABC is able to cut Acr-dG adducts quantitatively and specifically, and that the extent of UvrABC cutting at different sequences represents the extent of Acr-DNA adduct formation at the sequence.

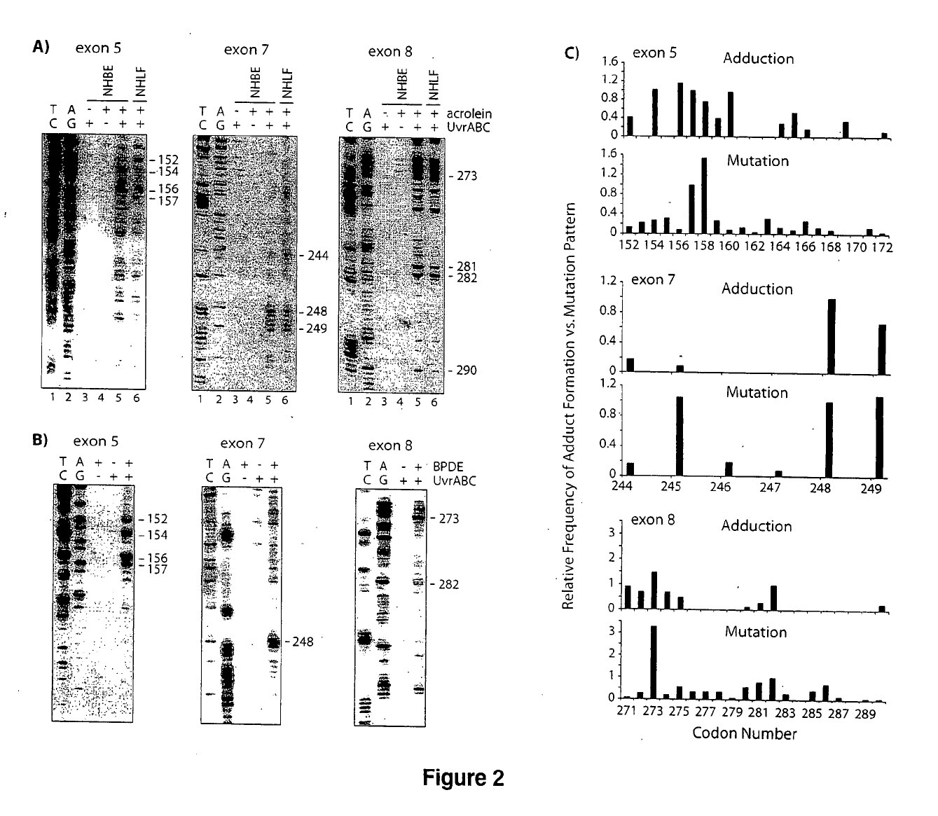

Acr-DNA Adducts Are Preferentially Formed at the Lung Cancer p53 Mutational Hotspots. Having established that the UvrABC nuclease is able to incise Acr-dG adducts specifically and quantitatively, we then used the UvrABC/LMPCR method to map the Acr-DNA binding spectrum in the coding strand of exons 5, 7 and 8 of the p53 gene in normal human lung cells. NHBE cells and NHLF were treated with different concentrations of Acr for 6 h, and genomic DNA was isolated to map the Acr-DNA binding pattern. For comparison purposes we also mapped the distribution of BPDE-DNA adducts in the p53 gene of NHBE cells treated with BPDE using the same UvrABC/LMPCR method. The results in FIG. 2A (lane 3-5) show that Acr-dG preferentially formed at codons 152, 154, 156, 157 and 158 in exon 5; codon 248 and 249 in exon 7; and codons 273 and 282 in exon 8 of the p53 gene. The Acr-DNA adduct distribution in exons 5, 7 and 8 of the p53 gene in the NHBE cells are very similar, but not quite identical, to the BPDE-DNA adduct distribution, as shown in FIG. 2B. BPDE preferentially forms DNA adducts at only CpG sites in codons 156, 157 and 158 of exon 5; codon 248 of exon 7; and codon 273 of exon 8 in thep53 gene in NHBE cells, which is consistent with previously published reports (5,6). Codons 157, 158, 248, 249, 273 and 282 of the p53 gene are the mutational hotspots in CS-related lung cancer and codons 249 and 273 of the p53 gene are mutational hotspot in lung cancers of both cigarette smokers and nonsmokers (FIG. 5). While Acr-dG adducts preferentially form at codon 249, BPDE-dG adducts do not (FIG. 2). It is worth noting that we have previously found that six of the activated cigarette PAHs either do not bind or weakly bind to this codon (Denissenko, M.-F., Pao, A., Tang, M.-s., Pfeifer, G.-P. (1996) Science 274, 430-432; Smith, L.-E. Denissenko, M.-F., Bennett, W.-P., Amin, S., Tang, M.-s., Pfeifer, G.-P. (2000) JNCI 92, 803-811).

We also mapped the distribution of Acr-DNA adducts along the p53 gene in NHLF treated with Acr. The results show that the Acr-DNA adduct distribution pattern in the p53 gene in NHLF was almost identical to that found in NHBE cells (FIG. 2A). These results clearly demonstrate that the specific binding spectrum of Acr in NHBE cells is due to the intrinsic binding specificity of Acr rather than the specificity of cell types.

The levels of Acr-dG formation in exons 5, 7 and 8 of the p53 gene of NHBE cells were compared with the mutation distribution in this gene in CS-related lung cancer obtained from the p53 database (FIG. 2C). The histograms of both show remarkable resemblance, which indicates that Acr-dG adducts may contribute to CS-related lung cancer and that Acr, instead of PAHs, from CS may be the etiological agent that causes mutations at codon 249 of the p53 in lung cancers of both cigarette smokers and nonsmokers. Acr is abundant in secondhand smoke and is also rich in cooking fumes (Hoffman, D., Hecht, S.-S. (1990) in Handbook of Experimental Pharmacology eds. Cooper, C.-S. & Grover, P.-L. (Springer-Verlag, Heidelberg), pp. 70-74; Gomes, R., Meek, M.-E., Eggleton, M. (2002) Concise International Chemical Assessment Document No. 43 World Health Organization, Geneva, Switzerland), which may be the major sources of Acr exposure for nonsmokers.

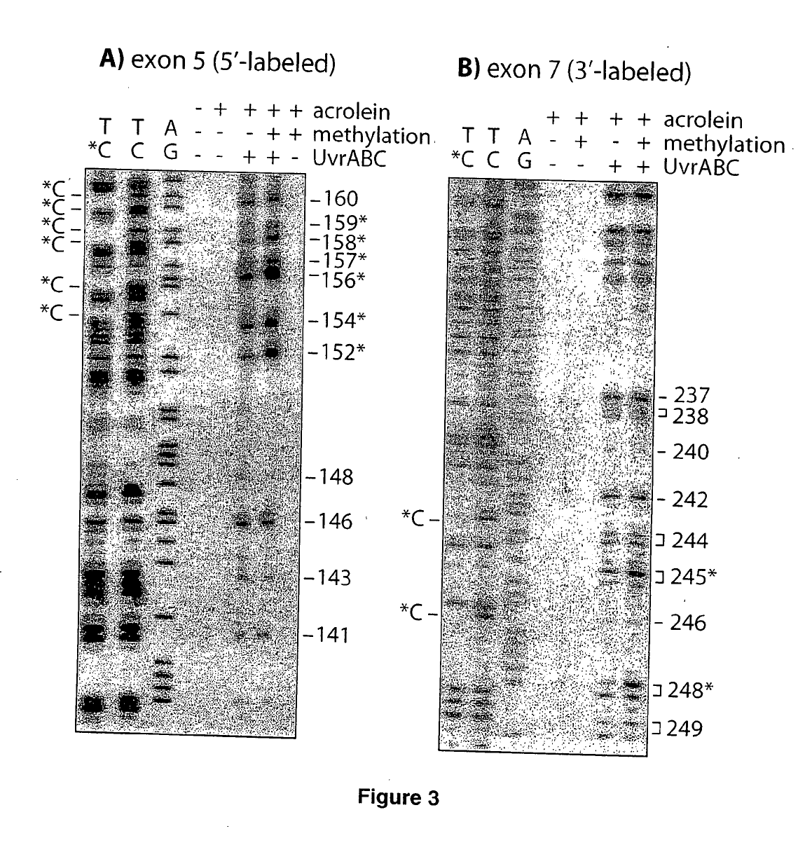

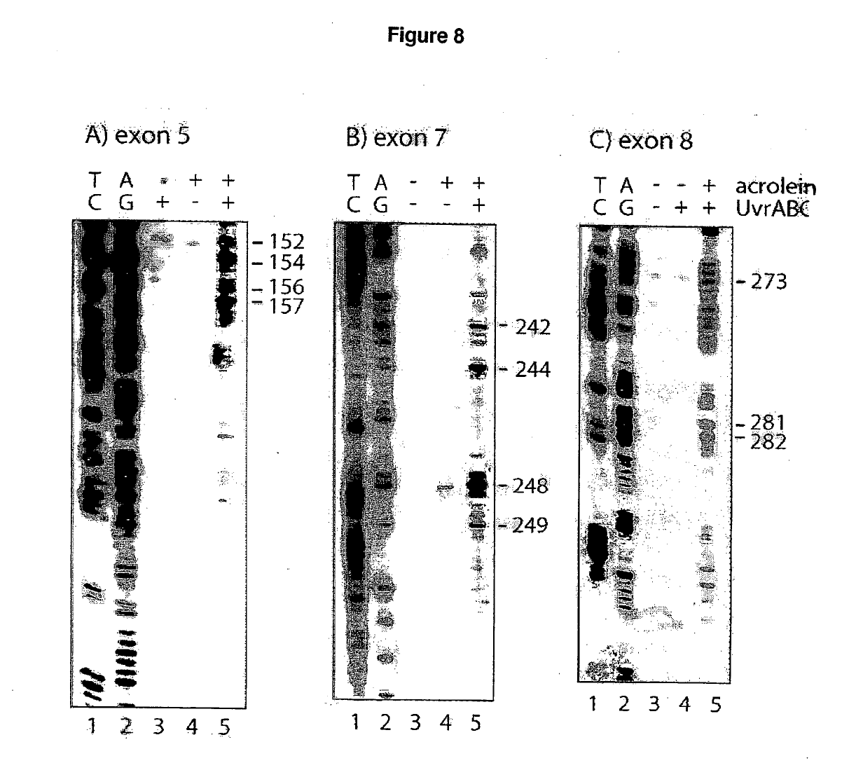

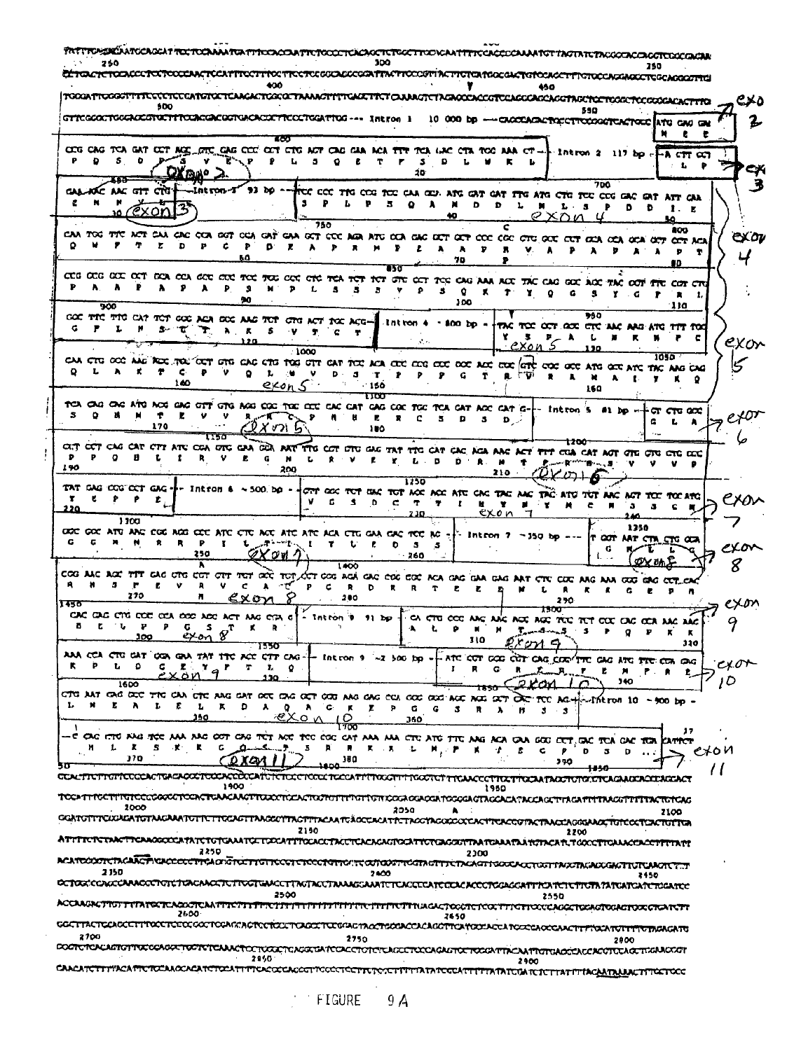

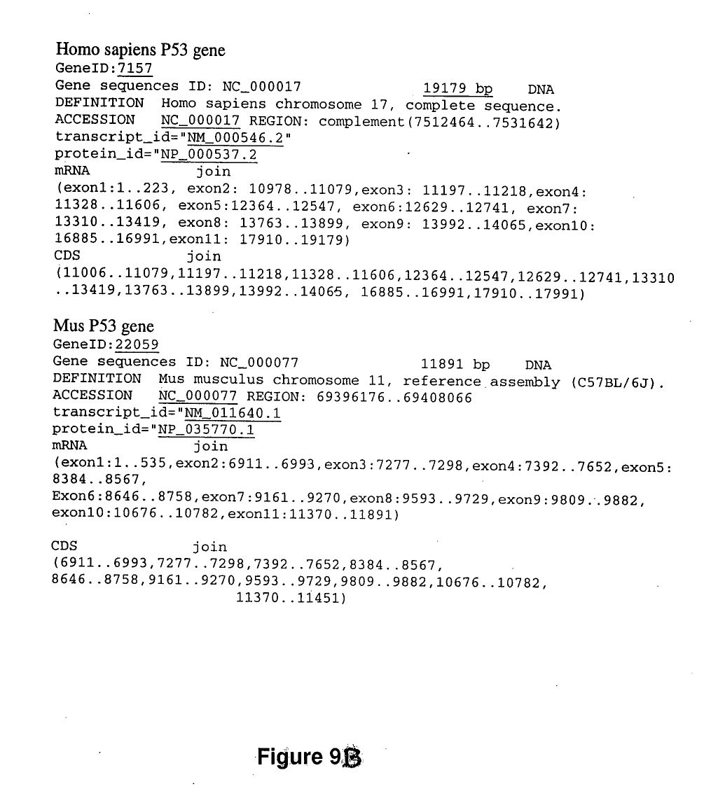

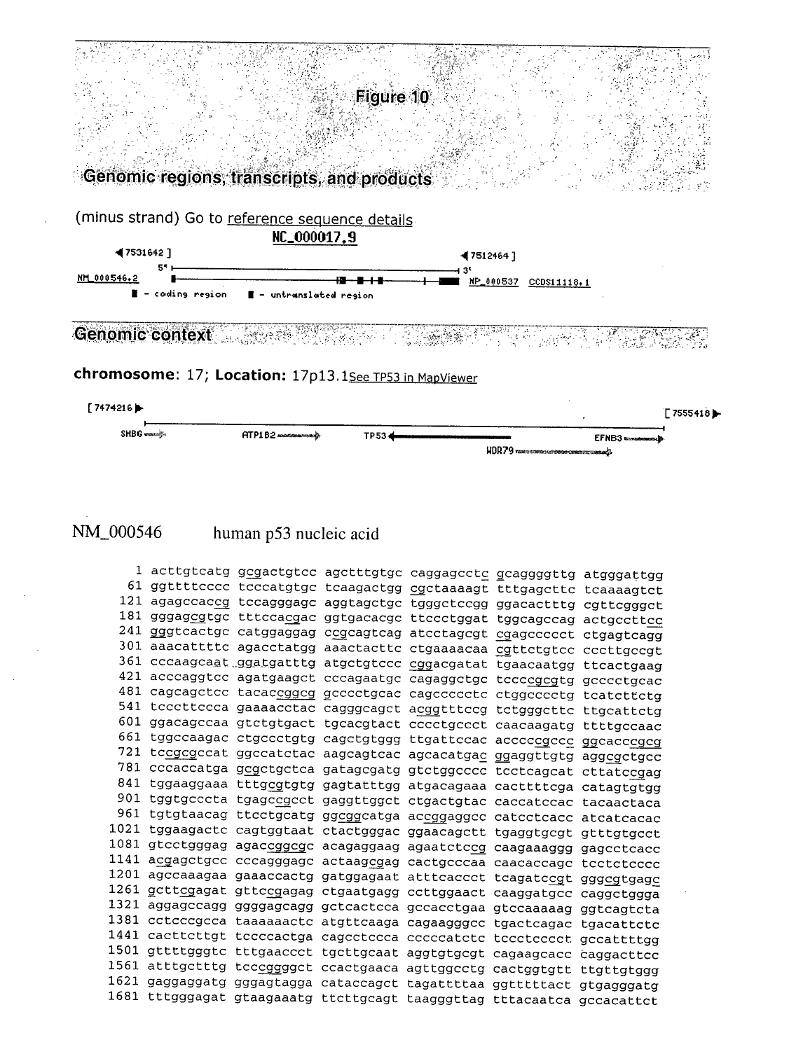

C5 Cytosine Methylation Enhances Acr-dG Binding at CpG Sites in the p53 Gene. Results in FIG. 2 show that, except for codon 249, all the preferential sites of Acr-dG binding are guanines within CpG sites. Why does Acr preferentially bind at codons containing CpG sequences? In human genomic DNA cytosines at CpG sequences are frequently methylated at the C5 position and this methylation may affect the stereostructure of DNA and/or nucleosomal structure in a manner that consequently affects bulky carcinogen binding (Tornaletti, S., Pfeifer, G.-P. (1995) Oncogene 10, 1493-1499; Johnson, W.-S., He, Q.-Y., Tomasz, M. (1995) Bioorg. Med. Chem. 3, 851-860; Chen, J.-X., Zheng, Y., West, M. & Tang, M.-s. (1998) Cancer Res. 58, 2070-2075). Indeed, we have found that 5C cytosine methylation at CpG sites enhances the binding of bulky chemicals at the adjacent guanines (Chen, J.-X., Zheng, Y., West, M. & Tang, M.-s. (1998) Cancer Res. 58, 2070-2075). Two approaches were undertaken to ascertain the reason behind the selectivity of Acr binding at CpG-containing codons in the p53 gene. First, we determined the Acr-dG binding pattern in the p53 gene by directly modifying NHBE genomic DNA and compared it to the pattern that resulted from treating intact NHBE cells; we found that both patterns of Acr-dG formation in the p53 gene are very similar, if not identical (FIG. 8). These results thus rule out nucleosomal structure playing a major role in determining the Acr-dG binding pattern in the p53 gene. Second, we determined the Acr-binding pattern in p53 DNA fragments with or without 5C cytosine methylation at CpG sequences. 3′-³²P-end-labeled DNA fragments of exon 7 and 5′-³²P-end-labeled DNA fragments of exon 5 of the p53 gene obtained by PCR amplification were subjected to SssI methylase treatment in the presence of 5-adenosylmethionine to methylate all cytosines at CpG sites. DNA fragments with and without methylation treatment were then modified with Acr and the adduct distributions were mapped by the UvrABC incision method. The extent of cytosine methylation was determined by Maxam-Gilbert chemical cleavage reactions (Maxam, A.-M., Gilbert, W. (1980) Methods Enzymol. 65, 499-560). Since hydrazine is unable to modify 5-C-methylated cytosines, both the 5′- and 3′-phosphodiester bonds of each methylated cytosine are refractory to piperidine hydrolysis and no cytosine ladders are observed at methylated cytosines (Maxam, A.-M., Gilbert, W. (1980) Methods Enzymol. 65, 499-560). As shown in FIGS. 3A and 3B, under the methylation conditions we used, all of the CpG sites in the DNA fragments are methylated, and the intensities of the UvrABC incision bands of Acr-DNA adducts are enhanced 2- to 4-fold at almost all CpG-containing codons, such as codons 152, 154, 156, 157, 158 and 159 (FIG. 3A) and codons 245 and 248 (FIG. 3B) in methylated versus unmethylated p53 DNA fragments. In contrast, the intensities of UvrABC incision bands of Acr-DNA adducts did not change significantly at the non-CpG—containing codons in methylated DNA fragments. To rule out the possibility that this enhancement of UvrABC cutting at methylated CpG sites is due to UvrABC having a higher cutting efficiency towards Acr adducts at methylated CpG sites versus other non-CpG sequences, the kinetics of UvrABC cutting for methylated CpG sequences and other sequences was determined in methylated p53 exon 7 DNA fragments to determine if the kinetics of UvrABC incision was in fact different for methylated CpG sites versus other sequences. We found that UvrABC incision at all Acr-binding sites, in both methylated CpG sequences (codons 245 and 248) and other unmethylated sequences, to be a function of incubation time and to plateau after 30 min of incubation, which leads us to conclude that the enhancement of UvrABC cutting at methylated CpG sites is due to preferential Acr binding as opposed to preferential UvrABC cutting for Acr adducts at methylated CpG sites (FIG. 7). Taken together, these results suggest that the strong binding of Acr at CpG-containing codons in the p53 gene in human lung cells is due to 5C cytosine methylation at these sequences. The results also suggest that the weaker Acr-dG formation at codons such as 158, 175 and 245 that contain CpG sequences is likely due to cytosines at these sequences not being methylated.

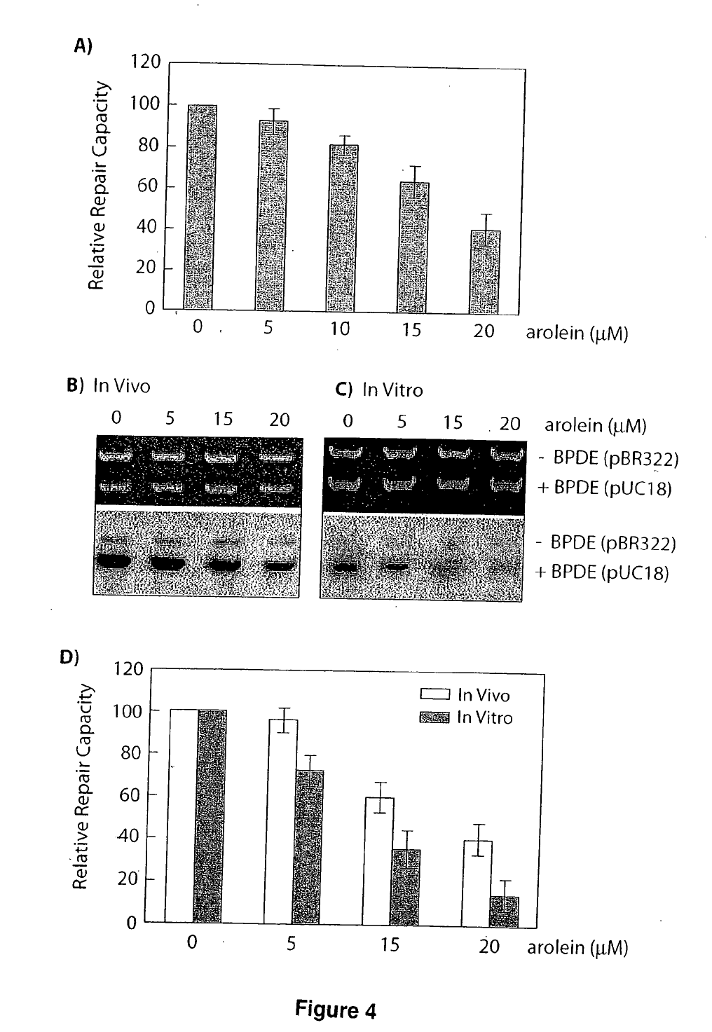

Acr Treatment Reduces the Ability of Cells to Repair BPDE-DNA Adducts. The carbonyl group and olefinic bond of Acr make this molecule reactive toward not only nucleic acids but also thiol-containing proteins (Gomes, R., Meek, M.-E., Eggleton, M. (2002) Concise International Chemical Assessment Document No. 43 World Health Organization, Geneva, Switzerland). It is conceivable that Acr binding may or may not affect the functions of the proteins. Previously, we have found aldehydes, such as 4-HNE and MDA, can greatly reduce cellular nucleotide excision repair (NER) capacity (Feng, Z., Hu, W., & Tang, M.-s. (2004) Proc. Nat. Acad. Sci. 101, 8598-8602; Feng, Z., Hu, W., Marnett, L., & Tang, M.-s. Mutat. Res. in press). We therefore determined the effect of Acr treatment on NER capacity using the well-established HCR assay and in vitro DNA damage-specific repair synthesis assay (Feng, Z., Hu, W., & Tang, M.-s. (2004) Proc. Nat. Acad. Sci. 101, 8598-8602; Feng, Z., Hu, W., Marnett, L., & Tang, M.-s. Mutat. Res. in press). We found that, similar to 4-HNE and MDA, Acr can greatly inhibit NER for BPDE-induced DNA damage (FIG. 4). This inhibitory effect is much more pronounced in cell lysates directly treated with Acr than whole cells treated with Acr (FIG. 4), suggesting that the Acr may interact with components in growth medium and that the cellular membrane may serve as a barrier to the uptake of Acr and that the inhibitory effect is due to the interactions of Acr with repair proteins.

Acr-dG Adducts Induce G to T Transversion Mutations in Human Cells. It has long been recognized that CS related lung cancers are rich in G to T transversions in the p53 gene (over 30% versus 10% in other human cancers) (Greenblatt, M.-S., Bennett, W.-P., Hollstein, M., Harris, C.-C. (1994) Cancer Res. 54, 4855-4878). This has been attributed to mutations induced by bulky carcinogens in CS, including PAHs (Denissenko, M.-F., Pao, A., Tang, M.-s., Pfeifer, G.-P. (1996) Science 274, 430-432; Denissenko, M., Pao, A., Pfeifer, G., Tang, M.-s. (1998) Oncogene 16, 1241-1249; Smith, L.-E. Denissenko, M.-F., Bennett, W.-P., Amin, S., Tang, M.-s., Pfeifer, G.-P. (2000) JNCI 92, 803-811; Pfeifer, G.-P., Hainaut, P. (2003) Mutat. Res. 526, 39-43). To determine the types of mutations induced by Acr-dG, we modified shuttle vector pSP 189 DNA containing the supF gene with Acr (1000M), and transfected the plasmid into NHLF for replication. Replicated plasmid was then transformed into indicator E. coli cells and mutant white colonies were collected and Acr-induced mutations in the supF gene were sequenced. We found that Acr modification resulted in greatly enhancing mutation frequency (from 4×10⁻⁴ to 120×10⁻⁴) and that more than 50% of the base substitutions induced by Acr-dG adducts are G to T transversions (28 out of 55 total base substitution mutants sequenced). These results further support the hypothesis that Acr in CS contributes significantly to p53 mutagenesis in lung cancer.

### Discussion

CS is the major cause of lung cancer deaths and 90% of all lung cancers in US are CS-related (Tobacco on Health (1997) World Health Organization, Geneva, Switzerland; Hoffmann, D., Hoffmann, I., El-Bayoumy, K. (2001) Chem. Res. Toxicol. 14, 767-790). CS contains more than 4000 compounds, many of which, including PAHs, N-nitrosamines, aromatic amines, and metals, are not only mutagenic but also well-established carcinogens in animal models (Hoffman, D., Hecht, S.-S. (1990) in Handbook of Experimental Pharmacology eds. Cooper, C.-S. & Grover, P.-L. (Springer-Verlag, Heidelberg), pp. 70-74; Hoffmann, D., Hoffmann, I., El-Bayoumy, K. (2001) Chem. Res. Toxicol. 14, 767-790; Hecht, S.-S., Carmella, S.-G., Murphy, S.-E., Foiles, P.-G., Chung, F.-L. (1993) J Cell Biochem. Suppl. 17F, 27-35). Therefore, it is reasonable to assume that these compounds contribute to CS-related lung carcinogenesis in humans. Previously, using the UvrABC/LMPCR method, we found that diol epoxides of potent PAH carcinogens found in CS preferentially form DNA adducts at p53 mutational hotspots in CS-related lung cancer, such as codons 156, 157, 158, 245, 273 and 282 (Denissenko, M.-F., Pao, A., Tang, M.-s., Pfeifer, G.-P. (1996) Science 274, 430-432; Smith, L.-E. Denissenko, M.-F., Bennett, W.-P., Amin, S., Tang, M.-s., Pfeifer, G.-P. (2000) JNCI 92, 803-811). In addition, BPDE-DNA adducts formed at these sites are poorly repaired (Denissenko, M., Pao, A., Pfeifer, G., Tang, M.-s. (1998) Oncogene 16, 1241-1249). Since the p53 gene is the most frequently mutated gene in CS-related lung cancer and the p53 mutational spectra in lung cancer of smokers and nonsmokers are distinctly different (Olivier, M., Eeles, R., Hollstein, M., Khan, M.-A., Harris, C.-C., Hainaut, P. (2002) Hum. Mutat. 19, 607-614. Greenblatt, M.-S., Bennett, W.-P., Hollstein, M., Harris, C.-C. (1994) Cancer Res. 54, 4855-4878) (FIG. 5), our findings strongly suggest that targeted DNA damage determines the p53 mutational spectrum in CS-related lung cancer. These results, however, do not exclude the possibility that many other CS compounds also contribute to lung carcinogenesis. In fact, these results raise the possibility that any DNA-damaging compounds present in CS that preferentially bind to the CS-related lung cancer p53 mutational hotspots are lung cancer etiological agents.

Acr is one of the most abundant compounds generated in CS; the amount of Acr in a single cigarette, depending on the manufacturer, ranges from 10 to 500 micrograms (Hoffman, D., Hecht, S.-S. (1990) in Handbook of Experimental Pharmacology eds. Cooper, C.-S. & Grover, P.-L. (Springer-Verlag, Heidelberg), pp. 70-74; Fujioka, K., Shibamoto, T. (2006) Environ. Toxicol. 21, 47-54). The total amount of PAHs present in a CS, in contrast, is in the range of just a few micrograms (Hoffman, D., Hecht, S.-S. (1990) in Handbook of Experimental Pharmacology eds. Cooper, C.-S. & Grover, P.-L. (Springer-Verlag, Heidelberg), pp. 70-74). Acr has been shown to interact with nucleophiles, including DNA and proteins in cell (Esterbauer, H., Schaur, R.-J., Zollner, H. (1991) Free Radic. Biol. Med. 11, 81-128). Unlike PAHs, where only metabolically activated forms can form adducts with DNA, Acr can directly interact with DNA and Conn DNA adducts (Nath, R.-G., Chen, H.-J.-C., Nishikawa, A., Young-Sciame, R. & Chung, F.-L. (1994) Carcinogenesis 15, 979-984; Tornaletti, S., Pfeifer, G.-P. (1995) Oncogene 10, 1493-1499). Similar to PAH-DNA adducts, Acr-DNA adducts induce mainly G:C to T:A transversion mutations (Curren, R.-D., Yang. L.-L., Conklin, P.-M., Grafstrom, R.-C., Harris, C.-C. (1988) Mutat. Res. 209, 17-22; Kawanishi, M., Matsuda, T., Nakayama, A., Takebe, H., Matsui, S., Yagi, T. (1998) Mutat. Res. 417, 65-73; Kanuri, M., Minko, I.-G., Nechev, L.-V., Harris, T.-M., Harris, C.-M. & Lloyd, R.-S. (2002) J. Biol. Chem. 21, 18257-18265; Yang, I.-Y., Chan, G., Miller, H., Huang, Y., Torres, M.-C., Johnson, F., Moriya, M. (2002) Biochemistry 41, 13826-13832), the major type of mutations found in p53 gene in CS-related lung cancer. Although the carcinogenicity of Acr in the lung has not been studied because of the severe toxicity associated with Acr treatment in animals, intraperitoneal injection of Acr has been shown to cause bladder cancer in rats (36). These results indicate that Acr is indeed a carcinogenic substance. While the effects of CS on Acr-dG formation in the lung tissue of cigarette smokers has not been determined, it has been reported that the level of Acr-dG DNA adducts in the oral tissue of smokers is in the range of a few micromoles per mole of guanine, which is far above the level of PAH-DNA adducts found in the oral or lung tissue of cigarette smokers (Nath, R.-G., Chung, F.-L. (1994) Proc. Nat. Acad. Sci. USA 91, 7491-7495; Nath, R.-G., Ocando, J.-E., Guttenplan, J.-B., & Chung, F.-L. (1998) Cancer Res. 58, 581-584). It is very likely that the level of Acr-dG in lung tissue is similar as that found in the oral tissues of cigarette smokers. If this is the case, then based on Acr abundance in CS, its reactivity towards DNA and mutagenicity of Acr-DNA adducts, Acr is potentially one of the major etiological agents of CS-induced lung cancer. Our current results demonstrate that, similar to PAHs, Acr preferentially binds to p53 mutational hotspots of CS-related lung cancer in human cells, including NHBE cells. These results strongly suggest that Acr-dG adducts, as well as PAH-DNA adducts, contribute greatly to these mutations in lung cancer.

We have found that all the Acr preferential binding guanines, except for codon 249, in the p. 53 gene are located at CpG sequences and that C5 cytosine methylation at CpG sites can greatly enhance Acr-dG adduct formation. This C5 cytosine methylation has been previously shown to also enhance guanine adduction at CpG sites by BPDE, aflatoxin B1 8,9-epoxide, and N-acetoxy-acetylaminofluorene, even though the binding positions by these agents on guanines are different (Nath, R.-G., Ocando, J.-E., Guttenplan, J.-B., & Chung, F.-L. (1998) Cancer Res. 58, 581-584). The precise mechanism of how C5 methylation enhances adduction to guanines at these sites has yet to be elucidated. It is worth noting that these CpG sites are in the coding region of the p53 gene, and are also very distant from promoter region. The function and the extent of methylation at these CpG sites in the p53 gene in NHBE and lung fibroblasts are also unknown and may vary among different individuals. If this is the case perhaps these variations may contribute to the different susceptibilities of individuals to CS-induced lung cancer.

We found that codon 249 in the p53 gene is a preferential binding site for Acr even though it is not a CpG-containing site and the Acr binding at this position is not affected by CpG methylation at the surrounding sequences (codon 248). Codon 249 is a mutational hot spot in lung and liver cancers (Greenblatt, M.-S., Bennett, W.-P., Hollstein, M., Harris, C.-C. (1994) Cancer Res. 54, 4855-4878) (FIG. 5). Intriguingly, we found that all the PAHs we tested and AFB1-8,9-epoxides, the etiological agents for liver cancer, do not preferentially bind to codon 249 (Hsu, I.-C., Metcalf, R.-A., Sun, T., Welsh, S.-A., Wang, N.-J., Harris, C.-C. (1991) Nature 350, 427-428; Denissenko, M.-F., Pao, A., Tang, M.-s., Pfeifer, G.-P. (1996) Science 274, 430-432; Smith, L.-E. Denissenko, M.-F., Bennett, W.-P., Amin, S., Tang, M.-s., Pfeifer, G.-P. (2000) JNCI 92, 803-811; Chen, J.-X., Zheng, Y., West, M. & Tang, M.-s. (1998) Cancer Res. 58, 2070-2075). Recently, however, we found that 4-HNE, another α,β-unsaturated aldehyde and a major lipid peroxidation product that is also able to interact with DNA to form exocyclic propanodeoxyguanosine adducts, preferentially forms DNA adducts at this codon (Hu, W., Feng, Z., Eveleigh, J., Iyer, G., Pan, J. Amin, S., Chung, F.-L., & Tang, M.-s. (2002) Carcinogenesis 23, 1781-1789). 4-HNE-dG adducts have been found to induce G:C to T:A mutations in human cells (Feng, Z., Hu, W., Amin, S., & Tang, M.-s. (2003) Biochemistry 42, 7848-7854). Increasing evidence suggests that lipid peroxidation and chronic oxidative stress play important roles in human carcinogenesis; while the mechanisms involved are unclear, it is possible that aldehydes resulting from endogenous lipid peroxidation and other sources, such as Acr, 4-HNE, MDA and crotonaldehyde, may contribute greatly to mutations at codon 249 in human cancer.

We have also found that Acr can greatly reduce the NER capacity. NER is the major repair pathway for bulky DNA damage, including PAH-DNA adducts and exocyclic propanodeoxyguanine adducts (Tang, M.-s. (1996) in Technologies for Detection of DNA Damage and Mutation ed. Pfeifer, G. (Plenum Press, New York) pp. 139-152; Sancar, A., Tang, M.-S. (1993) Photochem. Photobiol. 57, 905-921). It has been found that the NER capacity in individuals who have a genetic defect in NER genes, such as xeroderma pigmentosum patients, is reduced to 10% to 20% that of normal individuals; these patients have 2000- and 20- to 30-fold higher cancer incidence in skin and internal organs, respectively (Cleaver, J.-E. (2005) Nat. Rev. Cancer 5, 564-573). NER gene knockout animals have a predisposition for spontaneous and chemically-induced carcinogenesis (van Steeg, H., Mullenders, L.-H., Vijg, J. (2000) Mutat. Res. 450, 167-180). It has been strongly suggested that PAHs present in CS and in the environment are the agents responsible for the lung carcinogenesis, and the DNA damages induced by activated metabolites of PAHs initiate carcinogenesis (Denissenko, M.-F., Pao, A., Tang, M.-s., Pfeifer, G.-P. (1996) Science 274, 430-432; Smith, L.-E. Denissenko, M.-F., Bennett, W.-P., Amin, S., Tang, M.-s., Pfeifer, G.-P. (2000) JNCI 92, 803-811; Harvey, R.-G. (1991) In Chemistry and Carcinogenicity (Oxford University Press, United Kingdom) pp. 396). Our findings that Acr greatly inhibit cellular repair capacity to remove BPDE-DNA adducts in human lung cells strongly suggest that Acr also contributes significantly to lung carcinogenesis by its inhibitory effects on DNA repair in addition to damaging DNA directly. Our findings that the Acr-dG adduct distribution is similar to the mutational spectrum in CS-related lung cancer and that Acr can cause a significant inhibitory effect on DNA repair raise the possibility that Acr is an equally, if not more potent, lung cancer etiological agent as PAHs, considering the abundant amount of Acr in CS and in the environment. We propose that the carcinogenicity of Acr is derived from two detrimental effects: damaging DNA and reducing DNA repair capacity. These two effects may in turn lead to more mutations, which can be induced by both PAHs and Acr, to trigger carcinogenesis.

Worldwide, more than two million people die of CS-related cancer annually (Stewart, B.-W., Kleihues, P. (2003) World Cancer Report, IARC Press, World Health Organization). While smoking cessation is the most effective way to reduce these cigarette-induced deaths, this approach is unrealistic in the short term. Identifying the etiological agents for CS-induced cancer and designing methods to eliminate them from CS provides us with the simplest and most realistic solution. Although Acr carcinogenicity requires further epidemiological confirmation and studies in proper animal models, immediate measures to reduce this contaminant in both CS and in the environment seem warranted.

### Example 2

### Preparation and Testing of Filters for Removal or Sequestration of Acrolein from Combustion Products

In order to test whether a protein or nucleic acid, when attached to a filter, such as a cellulose filter, can aid in removal of acrolein from air or from tobacco smoke, the following studies will be conducted.

### Preparation of Target Proteins or Nucleic Acids

The proposed target proteins or nucleic acids, including the dinucleotide CpG or methylated cytosine may be prepared by methods known in the art. Custom synthesis of such proteins and nucleic acids may be done by laboratories such as ChemGene Corporation (see www.ChemGenes.com) or by Roche Diagnostics (see www.roche-diagnostics.com). Other proteins, such as albumin, may be purchased from a commercial source.

### Preparation of Filters

The following protocol is proposed for preparing nucleic acid-coated filters by covalently cross-linking nucleotides to free hydroxyl group-containing solid matrices.

Cellulose powder or cellulose fibers which contain free-hydroxyl groups may be used to covalently link oligonucleotides to a matrix to create a nucleic acid-containing, acrolein absorbing matrix. The procedure is as follows:

The matrix is incubated in 0.1 N NaOH for about 5 minutes at a rate of about 500 ml of 0.1 N NaOH for every 100 cm² of matrix material. The matrices are rinsed with about 1200 ml per 100 cm² of matrix of distilled water for 5 to 10 minutes, until the pH of the treated matrices became neutral. The matrix material is then dehydrated in methanol. Custom made purified oligonucleotides are solubilized in TE, pH8.0, to a concentration of 0.1% (W/V). One volume of this solution is then mixed with 4 volumes of a solution consisting of 50% by weight 1-cyclohexy-3-(2-morpholineothyl) carbondiimide metho-p-toluene solfonate (CMC) in 0.2 M sodium 2-(N-morpholino) ethanesulfonate, at pH 6.0. The treated solid matrices are submerged in the above mixture for about 12-20 hours at 20-24° C. The coated matrices are then rinsed 3 times with distilled water at 5 minute intervals, and then allowed to dry. This procedure is suitable for double stranded nucleic acid molecules.

Alternatively, it may be desired to attach single stranded nucleic acid molecules to a filter unit. This may be done by cross-linking the nucleic acid to the filter by using carbodiimide.

Single stranded nucleic acids molecules can be cross-linked to hydroxy group-containing matrices by water soluble carbodiimide in the following proposed protocol: Single stranded nucleic acids molecules may be custom made. The solid matrices described above are washed with methanol. A 0.1% by weight nucleic acid solution is mixed with 0.2M sodium 2-(N-morpholino) ethanesulfonate, pH6.0, at a ratio of about 6:1. Carboiimide is then added to final concentration of 7.2% W/W. 5000 cm² of solid matrices are submerged into 100 ml mixture described above. The resulting mixture is then incubated for 24 hours at 22° C. Following the incubation, the nucleic acid coated matrices are rinsed three times with distilled water, for about 5 minutes each rinse. The nucleic acid coated matrices are then allowed to dry at room temperature and stored at room temperature.

### Testing the Filter

The filters prepared above are then used to determine whether they could remove or sequester acrolein from a solution. For example, the cellulose filter fibers are spread out into a swatch of about 2 inches by 2 inches and then coated with various amounts of the nucleic acid preparations noted above or with a vehicle control. The treated fibers are dried overnight. Both control cellulose filters and cellulose filters to which has been attached the oligonucleotides, or CpG, or methylated cytosine, or a proposed protein or protein target may be used. The solution containing acrolein is then passed through the filters slowly to allow the reaction to occur between the filter containing the protein or nucleic acid and the acrolein in solution. The solution is collected after passing through the filter (the filtrate) and may be assayed using any of the procedures described above (eg, by measuring the ability of the solution to form DNA adducts or to inhibit DNA repair).

Alternatively, one may also test the material after filtering using an Ames test as described by Maron and Ames (Maron D M and Ames B N. 1983). Revised methods for the Salmonella mutagenicity assay. Mutation Research 113:173-215). Briefly, Salmonella strain TA98 is cultured overnight at 37 Cin Oxoid nutrient broth #2, incubated with serial dilutions of the filtered materials as noted above and rat liver S9 microsomal nucleases, in triplicate for 30 minutes at 37 C. The bacteria are then plated on minimal glucose plates. After a 48 hour incubation period at 37 C, the number of revertant mutants on each plate is counted. Tester strain TA 98 detects frameshift mutations, such as those generated by aromatic primary amines. Mutagens in the sample are detected as the number of bacteria induced to revert to their wild-type phenotype.

What is claimed is:

1. A method of preventing or inhibiting mutagenesis in a cell or tissue, containing a p53 gene, or a fragment thereof, wherein said gene or fragment thereof is reactive with an exogenous toxic agent, said method comprising contacting said cell or tissue with a composition comprising a second agent that blocks the reactivity of said exogenous toxic agent with a p53 gene, or a target sequence, or fragment thereof within the p53 gene, wherein said reactivity results in an increase in DNA adduct formation, and wherein said contacting prevents the binding of the exogenous toxic agent to the p53 gene, or the target sequence in the p53 gene, thereby reducing or inhibiting DNA adduct formation, wherein the target sequence or fragment thereof is present in exons 5, 7 and 8 of the p53 gene.

2. A method for removing, neutralizing, or sequestering an exogenous toxic agent from a liquid, wherein said exogenous toxic agent is reactive with a p53 gene or fragment thereof, comprising contacting said liquid with a composition comprising the nucleic acid encoding the p53 tumor suppressor, or a target sequence, or a fragment thereof within the p53 gene, that is reactive with, or binds to, the exogenous toxic agent.

3. A method for removing, neutralizing, or sequestering an exogenous toxic agent from a gaseous material, wherein said exogenous toxic agent is reactive with a p53 gene or fragment thereof, the method comprising contacting the gaseous material containing the exogenous toxic agent with a filter comprising a polymeric material derivatized with the nucleic acid encoding the p53 gene, or the p53 gene product, or a target sequence, or a fragment thereof within the p53 gene, that is reactive with, or binds to, the exogenous toxic agent.

4. The method of any one of claim 1, 2, or 3, wherein the exogenous toxic agent is acrolein.

5. The method of claim 3, wherein the polymeric material is selected from the group consisting of cellulose, starch and agarose.

6. The method of claim 1, wherein the target sequence is an unmethylated or methylated nucleobase, or an unmethylated or methylated dinucleotide, or a combination thereof.

7. The method of claim 6, wherein the methylated nucleobase is a cytosine.

8. The method of claim 6, wherein the methylated dinucleotide is CpG.

9. The method of claim 8, wherein the methylated CpG is present at a location in either the promoter region or the coding region of the p53 gene.

10. The method of claim 9, wherein the methylated CpG is found at a location selected from the group consisting of codons 152, 154, 156, 157, 158 of exon 5; codon 248 of exon 7, codons 273 and 282 of exon 8 of the p53 gene, and combinations thereof.

11. The method of claim 6, wherein the unmethylated nucleobase is found in codon 249 in exon 7 of the p53 gene.

12. A method of screening for a candidate compound that prevents the binding of a p53 tumor suppressor inhibitor to a p53 molecule, wherein said binding results in abrogation of the tumor suppressing activity or function of the p53 molecule, the method comprising:
(a) contacting the p53 molecule, or fragments thereof, or genomic DNA comprising the p53 molecule, with a candidate compound in the presence or absence of a known inhibitor, wherein said p53 molecule has the nucleic acid sequence of SEQ ID NO: 1 or SEQ ID NO: 3 and/or the amino acid sequence of SEQ ID NO: 2 or SEQ ID NO: 4; and
(b) determining the level of p53 expression or activity/function in the presence or absence of the candidate compound;
wherein the candidate compound is considered to be effective if the level of p53 expression or activity/function is higher in the presence of the candidate compound as compared to in the absence of the candidate compound.

13. The method of claim 12, wherein the determining of said p53 expression or activity/function is achieved by a method selected from the group consisting of reverse transcription-polymerase chain reaction (RT-PCR), real time PCR, northern blot analysis, in situ hybridization, cDNA microarray, electrophoretic gel analysis, an enzyme immunoassay (ELISA assays), a Western blot, a dotblot analysis, a protein microarray, a flow cytometric technique and proteomics analysis.

14. The method of claim 12, further comprising:
(c) treating a tumor bearing animal with a candidate compound capable of increasing the level of expression or function of the p53 molecule, and assessing the effect of the candidate compound on the growth, progression or metastasis of the tumor,
wherein said tumor arises as a result of loss of function of the p53 molecule or wherein the p53 molecule is mutated as a result of exposure of a cell in said animal to a mutagen;
and wherein a candidate compound effective at inhibiting the growth, progression, or metastasis of a tumor in said animal is identified as a positive candidate compound.

15. The method of claim 12, wherein the fragments are derived from exons 5, 7, or 8 of the p53 gene.

16. The method of claim 15, wherein the fragments contain one or more unmethylated or methylated cytosines.

17. The method of claim 16, wherein the methylated cytosines are present in a CpG dinucleotide.

18. The method of claim 17, wherein the methylated cytosines are present at a location selected from the group consisting of codons 152, 154, 156, 157, and 158 of exon 5, codon 248 of exon 7, codons 273 and 282 of exon 8 of the p53 gene, and combinations thereof.

19. The method of claim 16, wherein said unmethylated cytosine is present in codon 249 of exon 7 of the p53 gene.

20. A method of screening for a candidate compound capable of inhibiting the binding of a mutagenic agent to a p53 molecule, or to genomic DNA containing a p53 molecule, or to a fragment, nucleobase or dinucleotide derived therefrom, said method comprising:
(a) contacting the p53 molecule, or a fragment, nucleobase or dinucleotide derived therefrom, or genomic DNA containing the p53 gene, with a known mutagenic agent in the absence and presence of a candidate compound, wherein said p53 molecule is:
(i) a DNA corresponding to SEQ ID NO: 1 or SEQ ID NO: 3, and wherein the nucleic acid fragment, nucleobase, or dinucleotide is obtained from exons 5, 7 or 8 of the p53 gene of SEQ ID NO: 1 or SEQ ID NO: 3;
(ii) a protein comprising SEQ ID NO: 2 or SEQ ID NO: 4, or a fragment derived therefrom;
(vii) a nucleic acid comprising a sequence hybridizable to SEQ ID NO: 1 or SEQ ID NO: 3 or a complement thereof under conditions of high stringency, or a protein comprising a sequence encoded by said hybridizable sequence; or
(viii) a nucleic acid at least 90% homologous to SEQ ID NO: 1 or SEQ ID NO: 3 or a complement thereof as determined using an NBLAST algorithm or a protein encoded thereby;
(e) determining whether or not the candidate compound blocks the mutagenic effect of the known mutagenic agent on the p53 molecule.

21. The method of claim 20, wherein the effect of a known mutagenic agent on the p53 molecule is measured by assessing the formation of one or more DNA adducts in the p53 gene or a fragment thereof.

22. The method of claim 21, wherein the formation of a DNA adduct is measured by a UVR-BC/LMPCR method.

23. The method of claim 20, further comprising,
(f) treating a tumor bearing animal with said candidate compound and assessing the effect of the candidate compound on the growth, progression or metastasis of the tumor, wherein said tumor arises as a result of loss of function of the p53 molecule or wherein the p53 molecule is mutated as a result of exposure of a cell in said animal to mutagen;
wherein a candidate compound effective at inhibiting the growth, progression, or metastasis of a tumor in said animal is identified as a positive candidate compound.

24. The method of claim 20, wherein the fragment contains one or more unmethylated or methylated cytosines.

25. The method of claim 24, wherein the methylated cytosines are present in a CpG dinucleotide.

26. The method of claim 24, wherein the methylated cytosines are present at a location selected from the group consisting of codons 152, 154, 156, 157, and 158 of exon 5, codon 248 of exon 7, codons 273 and 282 of exon 8 of the p53 gene, and combinations thereof.

27. The method of claim 24, wherein said unmethylated cytosine is present at a location in codon 249 of exon 7.

28. A method for reducing the level of acrolein present in air containing combustion products by passing said air through a filter element capable of removing said acrolein present in said air, wherein said filter element comprises a polymer derivatized with a sequence obtained from the nucleic acid encoding the p53 tumor suppressor, or a fragment thereof, that is reactive with, or binds to, acrolein.

29. The method of claim 28, wherein the nucleic acid sequence obtained from the p53 tumor suppressor gene, or a fragment that reacts with, or binds to, acrolein, is an urunethylated or methylated nucleobase, or an unmethylated or methylated dinucleotide, or a combination thereof.

30. The method of claim 29, wherein the methylated nucleobase is a cytosine.

31. The method of claim 29, wherein the methylated dinucleotide is CpG.

32. The method of claim 31, wherein the CpG is present at a location in either the promoter region or the coding region of the p53 gene.

33. The method of claim 31, wherein the CpG is found at a location selected form the group consisting of codons 152, 154, 156, 157, and 158 in exon 5; codon 248 in exon 7, codons 273 and 282 in exon 8 of the p53 gene, and combinations thereof.

34. The method of claim 29, wherein the unmethylated nucleobase is found at a location in codon 249 in exon 7 of the p53 gene.

35. The method of claim 28, wherein the air comprises the vapor generated by volatilization of cooking oils and greases.

36. The method of claim 28, wherein the air comprises smoke generated by mainstream tobacco smoke or from second-hand tobacco smoke.

37. The method of claim 36, wherein said mainstream tobacco smoke retains nicotine content and desirable flavor components after passage through said filter.

38. The method of claim 28, wherein said polymer is selected from the group consisting of cellulose, starch, agarose, and combinations thereof.

39. The method of claim 28, wherein said method is used to filter air in a tobacco smoke-generating device or in a tobacco smoke-containing environment selected from the group consisting of a cigarette, free-standing cigarette filter, pipe, cigar, air ventilation filter, gas mask, and face mask.

40. A device for reducing the level of acrolein present in air containing combustion products wherein said device comprises a filter element through which air passes, said filter element capable of removing acrolein present in said air, said filter element comprising a polymer derivatized with an agent containing an aldehyde reactive group.

41. The device of claim 40, wherein said device filters smoke generated from frying or grilling food products, or from mainstream tobacco smoke, or from second-hand tobacco smoke.

42. The device of claim 41, wherein said mainstream tobacco smoke retains nicotine content and desirable flavor components after passage through said filter.

43. The device of claim 40, wherein said polymer is selected from the group consisting of cellulose, starch, agarose, and combinations thereof.

44. A device for reducing the level of acrolein present in air containing mainstream or secondary tobacco combustion products, or acrolein generated by heating cooking oils, wherein said device comprises a filter element through which air passes, said filter element capable of removing acrolein present in said air, said filter element comprising an agent selected from the group consisting of an amino acid, a peptide or protein, a nucleic acid, an oligonucleotide, a polynucleotide, a dinucleotide, a methylated dinucleotide, a nucleobase, a methylated nucleobase, methylated CpG, and any other synthetic or naturally occurring compound reactive with an aldehyde group, and combinations thereof.

45. The device of claim 44, wherein the nucleic acid comprises the nucleotide sequence set forth in SEQ ID NO: 1.

46. The device of claim 44, wherein the methylated dinucleotide is CpG.

47. The device of claim 44, wherein the methylated nucleobase is a cytosine.

48. The device of claim 44, wherein the nucleic acid is obtained from exons 5, 7, or 8 of the p53 gene.

49. The device of claim 46, wherein the CpG is found at a location selected from the group consisting of codons 152, 154, 156, 157, and 158 in exon 5; codon 248 in exon 7, codons 273 and 282 in exon 8 of the p53 gene, and combinations thereof.

50. The device of either one of claim 40 or 44, wherein said device is selected from the group consisting of a cigarette, a free-standing cigarette filter, a pipe, a cigar, an air ventilation filter, an air conditioner filter, a gas mask, and a face mask.

51. A filter for removing acrolein from a combustion product, said filter comprising a polymeric substrate to which is attached a chemical group reactive with acrolein.

52. The filter of claim 51, wherein the polymeric substrate is selected from the group consisting of cellulose, starch and agarose.

53. The filter of claim 51, wherein the chemical group reactive with acrolein is found on an agent selected from the group consisting of an amino acid, a peptide or protein, a nucleic acid, an oligonucleotide, a polynucleotide, a dinucleotide, a methylated dinucleotide, a nucleobase, a methylated nucleobase, methylated CpG, and any other synthetic or naturally occurring compound reactive with an aldehyde group, and combinations thereof.

54. The filter of claim 51, wherein the nucleic acid comprises the nucleotide sequence set forth in SEQ ID NO: 1.

55. The filter of claim 53, wherein the methylated dinucleotide is CpG.

56. The filter of claim 53, wherein the methylated nucleobase is a cytosine.

57. The filter of claim 53, wherein the nucleic acid is obtained from exons 5, 7, or 8 of the p53 gene.

58. The filter of claim 55, wherein the CpG is found at a location selected from the group consisting of codons 152, 154, 156, 157, and 158 in exon 5; codon 248 in exon 7, codons 273 and 282 in exon 8 of the p53 gene, and combinations thereof.


SEQUENCE LISTING (7 sequences)

SEQ ID NO: 1 - DNA, 2629 bp, Homo sapiens
  CDS (252)..(1430)
acttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggtaggggcccacttcaccgtactaaccagggaagctgtccctcatgttgaattttctctaacttcaaggcccatatctgtgaaatgctggcatttgcacctacctcacagagtgcattgtgagggttaatgaaataatgtacatctggccttgaaaccaccttttattacatggggtctaaaacttgacccccttgagggtgcctgttccctctccctctccctgttggctggtgggttggtagtttctacagttgggcagctggttaggtagagggagttgtcaagtcttgctggcccagccaaaccctgtctgacaacctcttggtcgaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtggagctggaagggtcaacatcttttacattctgcaagcacatctgcattttcaccccacccttcccctccttctccctttttatatcccatttttatatcgatctcttattttacaataaaactttgctgcca

SEQ ID NO: 2 - protein, 393 aa, Homo sapiens [sequence omitted]

SEQ ID NO: 3 - DNA, 2149 bp, Mus musculus
agccttggcttacaaagactctgtcttaaaaatccaaaaagatggctatgactatctagctggataggaaagagcacagagctcagaacagtggcggtccacttacgataaaaacttaattctttccactctttatacttgacacagaggcaggagccctccgaatcggtttccacccattttgccctcacagctctatatcttagacgacttttcacaaagcgttcctgctgagggcaacatctcagggagaatcctgactctgcaagtccccgcctccatttcttgccctcaacccacggaaggacttgcccttacttgttatggcgactatccagctttgtgccaggagtctcgcgggggttgctgggattgggactttcccctcccacgtgctcaccctggctaaagttctgtagcttcagttcattgggaccatcctggctgtaggtagcgactacagttagggggcacctagcattcaggccctcatcctcctccttcccagcagggtgtcacgcttctccgaagactggatgactgccatggaggagtcacagtcggatatcagcctcgagctccctctgagccaggagacattttcaggcttatggaaactccttcctccagaagatatcctgccatcacctcactgcatggacgatctgttgctgccccaggatgttgaggagttttttgaaggcccaagtgaagccctccgagtgtcaggagctcctgcagcacaggaccctgtcaccgagacccctgggccagtggcccctgccccagccactccatggcccctgtcatcttttgtcccttctcaaaaaacttaccagggcaactatggcttccacctgggcttcctgcagtctgggacagccaagtctgttatgtgcacgtactctcctcccctcaataagctattctgccagctggtgaagacgtgccctgtgcagttgtgggtcagcgccacacctccagctgggagccgtgtccgcgccatggccatctacaagaagtcacagcacatgacggaggtcgtgagacgctgcccccaccatgagcgctgctccgatggtgatggcctggctcctccccagcatcttatccgggtggaaggaaatttgtatcccgagtatctggaagacaggcagacttttcgccacagcgtggtggtaccttatgagccacccgaggccggctctgagtataccaccatccactacaagtacatgtgtaatagctcctgcatggggggcatgaaccgccgacctatccttaccatcatcacactggaagactccagtgggaaccttctgggacgggacagctttgaggttcgtgtttgtgcctgccctgggagagaccgccgtacagaagaagaaaatttccgcaaaaaggaagtcctttgccctgaactgcccccagggagcgcaaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagctacctgaagaccaagaagggccagtctacttcccgccataaaaaaacaatggtcaagaaagtggggcctgactcagactgactgcctctgcatcccgtccccatcaccagcctccccctctccttgctgtcttatgacttcagggctgagacacaatcctcccggtcccttctgctgccttttttaccttgtagctagggctcagccccctctctgagtagtggttcctggcccaagttggggaataggttgatagttgtcaggtctctgctggcccagcgaaattctatccagccagttgttggaccctggcacctacaatgaaatctcaccctaccccacaccctgtaagattctatcttgggccctcatagggtccatatcctccagggcctactttccttccattctgcaaagcctgtctgcatttatccaccccccaccctgtctccctcttttttttttttttacccctttttatatatcaatttcctattttacaataaaattttgttatcact

SEQ ID NO: 4 - protein, 390 aa, Mus musculus
MTAMEESQSDISLELPLSQETFSGLWKLLPPEDILPSPHCMDDLLLPQDVEEFFEGPSEALRVSGAPAAQDPVTETPGPVAPAPATPWPLSSFVPSQKTYQGNYGFHLGFLQSGTAKSVMCTYSPPLNKLFCQLVKTCPVQLWVSATPPAGSRVRAMAIYKKSQHMTEVVRRCPHHERCSDGDGLAPPQHLIRVEGNLYPEYLEDRQTFRHSVVVPYEPPEAGSEYTTIHYKYMCNSSCMGGMNRRPILTIITLEDSSGNLLGRDSFEVRVCACPGRDRRTEEENFRKKEVLCPELPPGSAKRALPTCTSASPPQKKKPLDGEYFTLKIRGRKRFEMFRELNEALELKDAHATEESGDSRAHSSYLKTKKGQSTSRHKKTMVKKVGPDSD

SEQ ID NO: 5 - DNA, 1182 bp, Homo sapiens
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 6 - DNA, 2629 bp, Homo sapiens
acttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattggacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggtaggggcccacttcaccgtactaaccagggaagctgtccctcatgttgaattttctctaacttcaaggcccatatctgtgaaatgctggcatttgcacctacctcacagagtgcattgtgagggttaatgaaataatgtacatctggccttgaaaccaccttttattacatggggtctaaaacttgacccccttgagggtgcctgttccctctccctctccctgttggctggtgggttggtagtttctacagttgggcagctggttaggtagagggagttgtcaagtcttgctggcccagccaaaccctgtctgacaacctcttggtcgaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtggagctggaagggtcaacatcttttacattctgcaagcacatctgcattttcaccccacccttcccctccttctccctttttatatcccatttttatatcgatctcttattttacaataaaactttgctgcca

SEQ ID NO: 7 - protein, 393 aa, Homo sapiens [sequence omitted]